SEQUENCE LISTING (6 sequences)

SEQ ID NO: 1 - DNA, 19 bp, Artificial sequence
  Single strand DNA oligonucleotide
gtgccagcmgccgcggtaa

SEQ ID NO: 2 - DNA, 20 bp, Artificial sequence
  Single strand DNA oligonucleotide
ggactachvgggtwtctaat

SEQ ID NO: 3 - DNA, 19 bp, Artificial sequence
  Single strand DNA oligonucleotide
gccaccggcttcagaaaaa

SEQ ID NO: 4 - DNA, 20 bp, Artificial sequence
  Single strand DNA oligonucleotide
ccggaagctattcgcagatt

SEQ ID NO: 5 - DNA, 21 bp, Artificial sequence
  Single strand DNA oligonucleotide
tcagtcaacgggggacataaa

SEQ ID NO: 6 - DNA, 21 bp, Artificial sequence
  Single strand DNA oligonucleotide
ggggctgtactgcttaaccag

(19) United States
(12) Patent Application Publication
(10) Pub. No.: US 2023/0330051 A1
(43) Pub. Date: Oct. 19, 2023

(54) METHOD FOR MODULATING WEIGHT

(71) Applicant: Yeda Research and Development Co. Ltd., Rehovot (IL)

(72) Inventors: Eran ELINAV, Mazkeret Batya (IL); Leviel FLUHR, Rehovot (IL); Uria MOR, Rehovot (IL)

(73) Assignee: Yeda Research and Development Co. Ltd., Rehovot (IL)

(21) Appl. No.: 18/027,168

(22) PCT Filed: Sep. 20, 2021
(86) PCT No.: PCT/IL2021/051146

§ 371 Date: Mar. 20, 2023

(30) Foreign Application Priority Data
Sep. 21, 2020 (IL) 277488

(51) Int. Cl. A61K 31/201; A61K 31/198; A61K 31/465; A61K 45/06; A23G 4/12

(52) U.S. Cl. CPC A61K 31/201; A61K 31/198; A61K 31/465; A61K 45/06; A23G 4/12

(57) ABSTRACT

A method of treating obesity in a subject in need thereof is disclosed. The method comprises administering to the subject a therapeutically effective amount of an agent that specifically increases the amount of hexadecadienoate (16:2n6), N-acetylglycine, 1-palmitoyl-2-gamma-linolenoyl-GPC (16:0/18:3n6) and/or Hexanoylglycine; or an agent that decreases the amount of dimethylglycine (DMG) in the fecal metabolome of the subject. Agents suitable for same are also disclosed.

### RELATED APPLICATIONS

This application claims the benefit of priority of Israeli Patent Application No 277488 filed 21 Sep. 2020 the contents of which are incorporated herein by reference in their entirety.

### SEQUENCE LISTING STATEMENT

The ASCII file, entitled 89027 Sequence Listing.txt, created on 16 Sep. 2021, comprising 4,096 bytes, submitted concurrently with the filing of this application is incorporated herein by reference.

### FIELD AND BACKGROUND OF THE INVENTION

The present invention, in some embodiments thereof, relates to methods of modulating weight and, more particularly, but not exclusively, to reduction of weight following smoking cessation.

Cigarette smoking is considered a major public health concern and the leading cause of preventable death worldwide. In 2015, over 1.1 billion people smoked tobacco, with the prevalence of smoking reported to be higher in males compared to females. Smoking rates have been declining in developing countries, but remain high among youth, while tobacco consumption continues to rise in developing countries. It is estimated that smoking contributes 1 trillion dollars a year to the global health costs, including induction of a higher risk of development of numerous cancer types including lung, liver, pancreas, stomach and colon, ischemic heart disease, cerebrovascular events, aortic aneurysm, peripheral arterial disease, chronic obstructive pulmonary disease (COPD), osteoporosis, and age-related macular degeneration. Additionally, smoking is associated with chronic inflammation and compromised immunity, leading to a propensity to develop pneumonia, exacerbated tuberculosis, and other airway infections.

Smoking cessation has a beneficial impact on most of the above disease risks and health conditions. In general, 70% of active long-term smokers express a desire to quit, and 50% report an attempt to quit within the past year. However, smoking cessation involves significant short-term and long-term adverse effects, including increased anger/irritability, anxiety, depression, impatience, trouble sleeping, restlessness, and difficulty in concentrating. Other withdrawal symptoms may include constipation, cough, dizziness, drowsiness, headache, impulsivity, fatigue, flu-like symptoms, mood swings and mouth ulcers.

However, the most prohibitive quitting-related adverse effect and the leading cause of refusal to quit or failed quitting is smoking cessation-associated weight gain (SCWG). Indeed, smoking cessation leads to an average weight gain of 4-5 kg, even under conditions of stable total caloric intake. Several mechanisms have been proposed to explain this metabolic phenomenon, such as increased energy efficiency, decreased resting metabolic rate, decreased physical activity and increased lipoprotein lipase activity. For example, while nicotine increases the basal metabolic rate during active smoking, its washout during cessation may predispose to weight gain if ex-smokers maintain a pre-cessation caloric consumption. Nicotine is also suggested to interfere in hypothalamic activity and contribute to a decrease in appetite, while smoking cessation may lead to opposite effects driving an increased food intake resulting in weight gain. Additionally, ex-smokers feature a lower ability to perceive fat and sweetness, thereby deriving less pleasure from foods, leading to an increased preference for sweet-tasting foods which, over time, might lead to increased caloric intake and weight gain. Interestingly, food addiction appears to activate similar reward pathways in the brain as does smoking. However, several studies suggest that SCWG occurs even in the absence of enhanced caloric intake. Moreover, interventional attempts aiming at dietary or pharmacological induction of caloric restriction resulted in disappointing success rates in preventing or ameliorating SCWG.

Background art includes US Patent Application No. 20060078627 and US Patent Application No. 20120219621.

Additional background art includes McEntyre et al., Ann Clin Biochem. 2015;52(Pt 3):352-360; Magnusson M, et al., Diabetes. 2015;64(8):3010-3016; Salek et al., Physiol Genomics 29: 99-108, 2007; Zhao et al., Obesity, Science and Practice, 2016, pages 309-317; Moore et al., Metabolomics : Official Journal of the Metabolomic Society. 2014 April;10(2):259-269. Murphy et al., J Gerontol A Biol Sci Med Sci. 2017 October; 72(10): 1352-1359.

### SUMMARY OF THE INVENTION

According to an aspect of the present invention there is provided a method of treating obesity in a subject in need thereof, the method comprising administering to the subject a therapeutically effective amount of an agent that specifically increases the amount of hexadecadienoate (16:2n6), N-acetylglycine, 1-palmitoyl-2-gamma-linolenoyl-GPC (16:0/18:3n6) and/or Hexanoylglycine; or an agent that decreases the amount of dimethylglycine (DMG) in the fecal metabolome of the subject, thereby treating the obesity.

According to an aspect of the present invention there is provided a method of reducing the risk of weight gain following nicotine smoking cessation in a subject comprising administering to the subject an effective amount of an agent that specifically increases the amount of hexadecadienoate (16:2n6), N-acetylglycine, 1-palmitoyl-2-gamma-linolenoyl-GPC (16:0/18:3n6) and/or Hexanoylglycine; or an agent that decreases the amount of dimethylglycine (DMG) in the fecal metabolome of the subject, thereby reducing the risk of weight gain in the subject.

According to an aspect of the present invention there is provided a method of reducing the risk of weight gain following nicotine smoking cessation in a subject comprising administering to the subject an effective amount of a fecal transplant derived from a healthy, non-smoker, thereby reducing the risk of weight gain in the subject.

According to an aspect of the present invention there is provided a method of analyzing the likelihood of weight gain in a subject on cessation of nicotine smoking, comprising analyzing the amount of hexadecadienoate (16:2n6), N-acetylglycine, 1-palmitoyl-2-gamma-linolenoyl-GPC (16:0/18:3n6), Hexanoylglycine and DMG in a fecal sample of the subject wherein when the level of DMG is above a predetermined amount and/or the level of hexadecadienoate (16:2n6), N-acetylglycine, 1-palmitoyl-2-gamma-linolenoyl-GPC (16:0/18:3n6) or Hexanoylglycine is below a predetermined amount, it is indicative that the subject has a predisposition to weight gain on cessation of nicotine smoking.

According to an aspect of the present invention there is provided a method of reducing the risk of weight gain following nicotine smoking cessation in a subject comprising administering to the subject an effective amount of an antibiotic thereby reducing the risk of weight gain following nicotine smoking cessation.

According to an aspect of the present invention there is provided a chewing gum comprising a metabolite selected from the group consisting of hexadecadienoate (16:2n6), N-acetylglycine, 1-palmitoyl-2-gamma-linolenoyl-GPC (16:0/18:3n6) and/or Hexanoylglycine.

According to an aspect of the present invention there is provided a method of treating a disease associated with weight loss in a subject in need thereof, the method comprising administering to a subject a therapeutically effective amount of an agent which decreases the amount of hexadecadienoate (16:2n6), N-acetylglycine, 1-palmitoyl-2-gamma-linolenoyl-GPC (16:0/18:3n6) and/or Hexanoylglycine or an agent that specifically increases the amount of dimethylglycine (DMG) in the fecal metabolome of the subject, thereby treating the disease associated with weight loss.

According to an aspect of the present invention there is provided a use of an agent that specifically increases the amount of hexadecadienoate (16:2n6), N-acetylglycine, 1-palmitoyl-2-gamma-linolenoyl-GPC (16:0/18:3n6) and/or Hexanoylglycine; or an agent that decreases the amount of dimethylglycine (DMG) in the fecal metabolome of the subject for treating obesity or for reducing the risk of weight gain following nicotine smoking cessation.

According to an aspect of the present invention there is provided a use of a fecal transplant derived from a healthy, non-smoker for reducing the risk of weight gain in a subject.

According to an aspect of the present invention there is provided a use of an antibiotic for reducing the risk of weight gain following nicotine smoking cessation.

According to an aspect of the present invention there is provided a use of an agent which decreases the amount of hexadecadienoate (16:2n6), N-acetylglycine, 1-palmitoyl-2-gamma-linolenoyl-GPC (16:0/18:3n6) and/or Hexanoylglycine or an agent that specifically increases the amount of dimethylglycine (DMG) in the fecal metabolome of the subject, thereby treating the disease associated with weight loss.

According to further features in the described preferred embodiments, the agent comprises hexadecadienoate (16:2n6), N-acetylglycine, 1-palmitoyl-2-gamma-linolenoyl-GPC (16:0/18:3n6) and/or Hexanoylglycine.

According to further features in the described preferred embodiments, the agent that decreases the amount of DMG is an inhibitor of the DMG synthesis pathway.

According to further features in the described preferred embodiments, the agent that decreases the amount of DMG comprises a choline-poor diet.

According to further features in the described preferred embodiments, the administering is effected immediately following smoking cessation.

According to further features in the described preferred embodiments, the method further comprises recommending the subject to start a weight loss program if the subject is found predisposed to weight gain on cessation of smoking.

According to further features in the described preferred embodiments, the method further comprises administering to the subject an effective amount of a fecal transplant derived from a healthy, non-smoker.

According to further features in the described preferred embodiments, the chewing gum further comprises nicotine.

According to further features in the described preferred embodiments, the disease is selected from the group consisting of cancer, hyperthyroidism, cathexia and anorexia.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-D. Distinct metabolite alterations in smoking and smoking cessation induced by the host and its microbiome (A) A linear mixed model utilizing both smoking and antibiotics alterations throughout time and quantifying their interactive impacts on serum metabolite levels at the active smoking period (day 15). Venn diagram (left)-representing significant (p<0.05) metabolites impacted by smoking, antibiotic and their interactions. Post-hoc: statistical hypothesis testing with Tukey correction for multiple comparisons-SMK vs. NS mice (i), SMK vs. SMK+abx mice (ii), NS+abx vs. NS mice (iii), NS+abx vs. SMK+abx mice (iv). For the comparisons of i and ii, the metabolites of interest (significant) marked in purple, for the comparisons of iii and iv, the metabolites of interest (insignificant) marked in green (q<0.1). Venn diagram (right): representing metabolites fulfilling both the mixed linear model and statistical hypothesis testing criteria. (B) GLM regression (DESeq2) of EC levels on day 21 and weight change (day 35). Inset: betaine-reductase counts as a function of weight change. (C) Serum DMG levels of GF a week after FMT. Timepoint B: recipient mice receiving microbiomes from NS (n=9), SMK (n=11) mice; Mann Whitney U-test. (D) Schematic representation of shared host-microbiome DMG biosynthesis pathway, depicting quantification of key enzymes and metabolites in stool, serum and liver at the active smoking period. NS (n=7-9) and SMK (n=6-8) mice; Metabolites: 2-way ANOVA and BH correction (q<0.1). Symbols or horizontal lines represent the mean, error bars SEM or 10-90 percentiles. *p<0.05; **p<0.01; ***p<0.001; ****p<0.0001.

FIGS. 2A-F. Accumulation of dimethyl glycine and depletion of acetyl glycine drive smoking cessation-induced weight gain (A) Weight change upon PBS or DMG administration. PBS (n=30-40), DMG (n=30-40) supplemented mice; linear mixed model-p=0.0021. Day 21: Unpaired t-test. Inset: iAUC describing the weight change over time in the designated groups. Unpaired t-test. Results are pooled from 4 independent repeats. (B) Stool calories three weeks into PBS or DMG administration. PBS (n=9), DMG (n=7) supplemented mice; Unpaired t-test. (C) Weight change during active smoking and cessation. NS (n=30), SMK (n=29), NS+abx (n=29), SMK+abx (n=30), NS+abx+PBS (n=10), NS+abx+DMG (n=10), SMK+abx+PBS (n=19), SMK+abx+DMG (n=30) mice; Cessation: Day 42: One-way ANOVA and Sidak correction, (Inset: iAUC data for smoking and cessation, see FIG. 4A). Results are pooled from 3 independent repeats. (D) Weight change during active smoking and cessation during consumption of a Choline-deficient diet (CDD). NS (n=20), SMK (n=20), NS+abx (n=20), SMK+abx (n=19) mice; Cessation: linear mixed model- p=0.004. Day 35: One-way ANOVA and Sidak correction. Inset: iAUC describing the weight change at active smoking or cessation. One-way ANOVA and Sidak correction. Results are pooled from 2 independent repeats. (E) Weight change under HFD or ACG consumption. HFD (n=25-30), ACG (n=24-29) consuming mice; Day 42: Unpaired t-test. Inset: iAUC describing the weight change at active smoking or cessation. Unpaired t-test. Results are pooled from 3 independent repeats. (F) Weight change during active smoking and cessation upon HFD or ACG consumption. NS (n=10), SMK (n=10), NS+abx (n=10), SMK+abx (n=10) mice; Cessation: 3way ANOVA- p<0.001. Day 35: One-way ANOVA and Sidak correction. Inset: iAUC describing the weight change during active smoking or cessation. One-way ANOVA and Sidak correction. Gray background in graphs depicts the cessation period. Symbols or horizontal lines represent the mean, error bars SEM or 10-90 percentiles. *p<0.05; **p<0.01; ***p<0.001; ****p<0.0001.

FIGS. 3A-H. Metabolic consequences of Dimethylglycine supplementation to naïve mice (A-D) Weight change upon PBS or DMG administration via osmotic pumps. PBS (n=10), DMG (n=10) supplemented mice; Final experiment day: Unpaired t-test. Inset: iAUC describing the weight change at active smoking or cessation. Unpaired t-test. (A-C) Experiments started at 10-week old mice, (D) Experiments started at 7-week old mice. (E-H) Metabolic cage analysis over a period of 172 hours. PBS (n=4-8), DMG (n=4-8) supplemented mice. Locomotion activity (E), FI Total Kcal (F), RER (G) and energy expenditure (H); Inset: AUC; Mann Whitney U-test. Gray background in graphs depict the dark cycle. Symbols or horizontal lines represent the mean, error bars SEM or 10-90 percentiles. *p<0.05; **p<0.01; ***p<0.001; ****p<0.0001.

FIGS. 4A-I. Metabolic consequences of Dimethylglycine supplementation to smoking cessation mice (A) iAUC describing the weight change at active smoking or cessation of FIG. 5C. NS (n=30), SMK (n=29), NS+abx (n=29), SMK+abx (n=30), NS+abx+PBS (n=10),

NS+abx+DMG (n=10), SMK+abx+PBS (n=19), SMK+abx+DMG (n=30) mice; One-way ANOVA and Sidak correction. (B-C) Weight change during active smoking and cessation (B). NS (n=10), SMK (n=10), NS+abx (n=9), SMK+abx (n=10), NS+abx+PBS (n=10), NS+abx+DMG (n=10), SMK+abx+PBS (n=9-10), SMK+abx+DMG (n=10) mice; Cessation Day 42: One-way

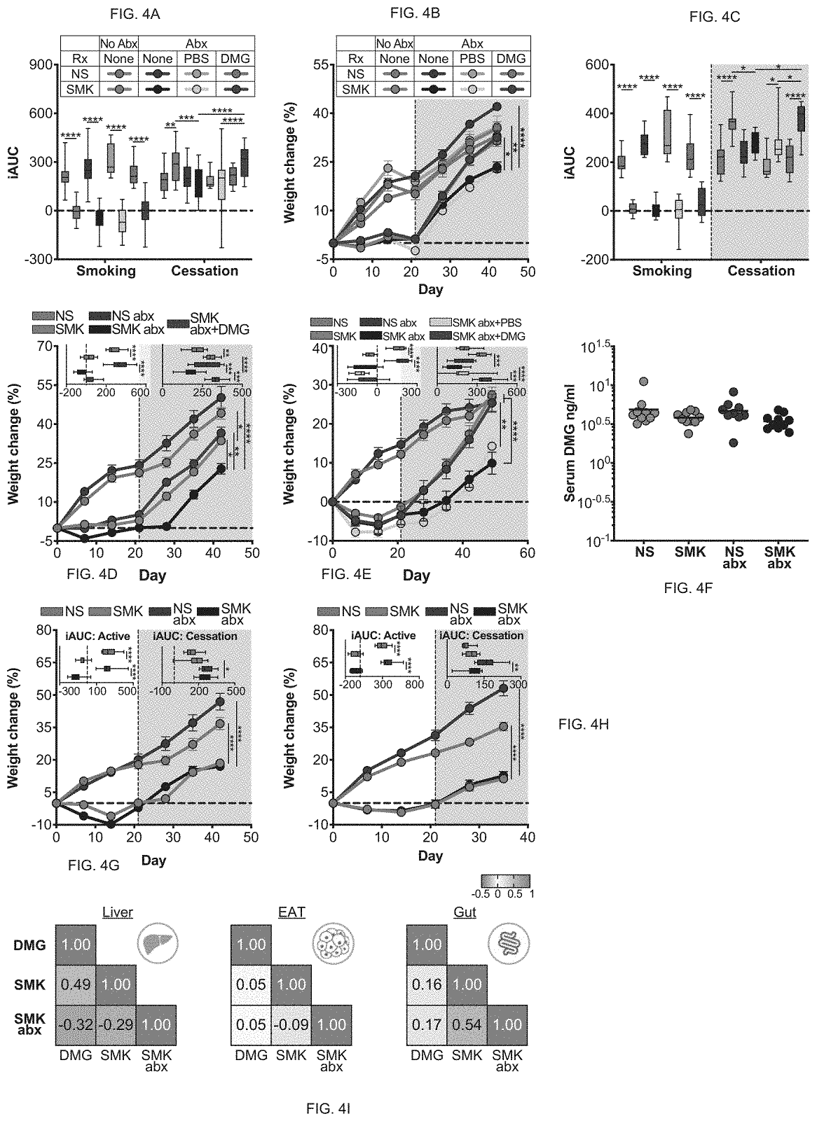

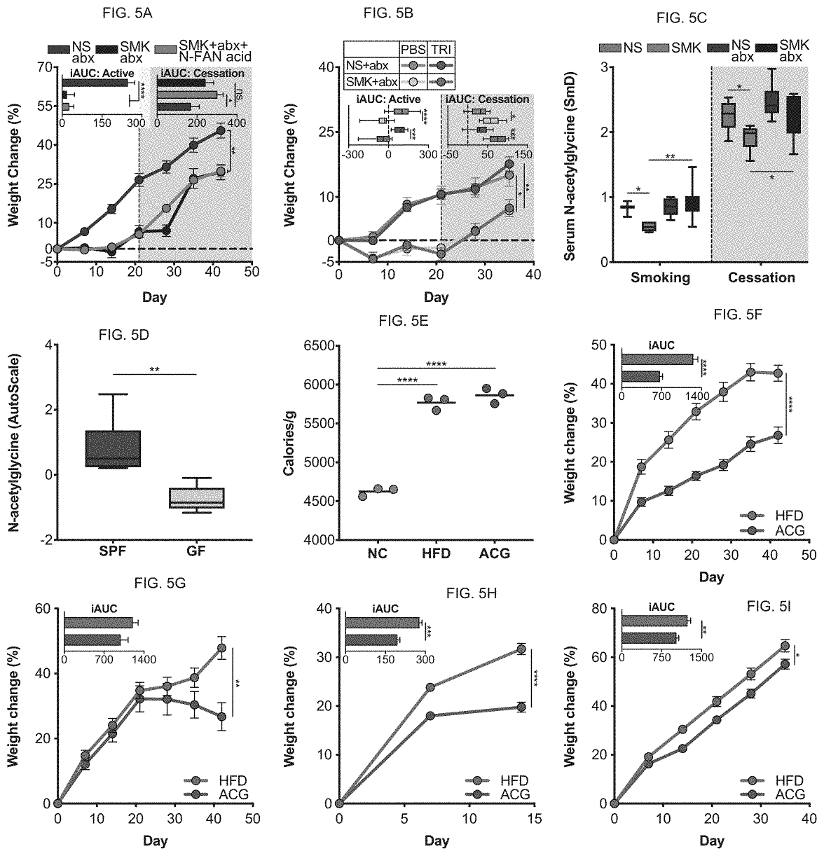

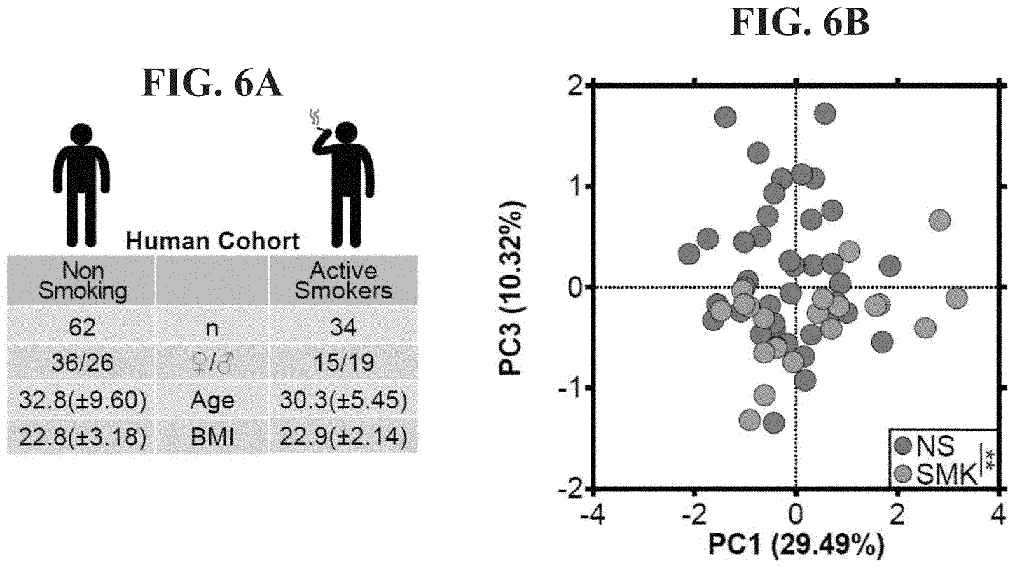

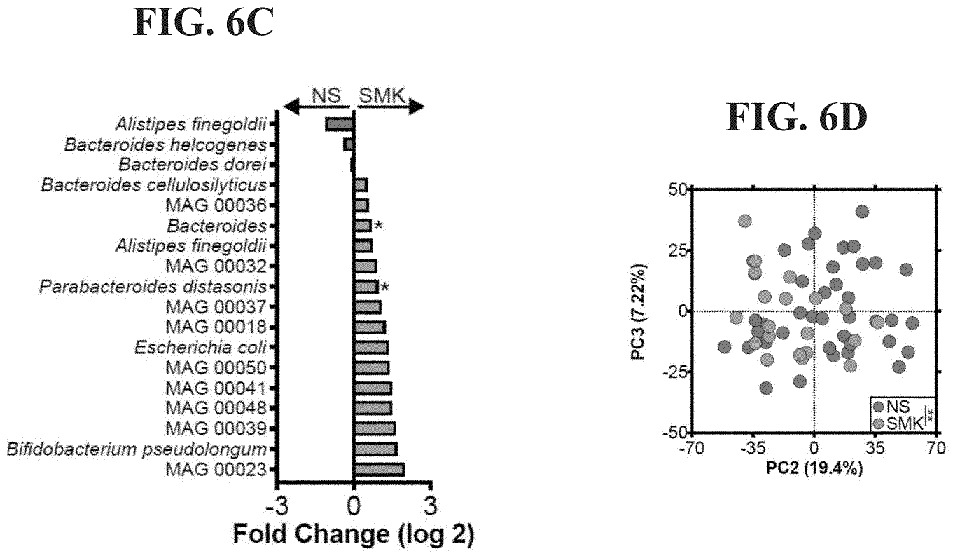

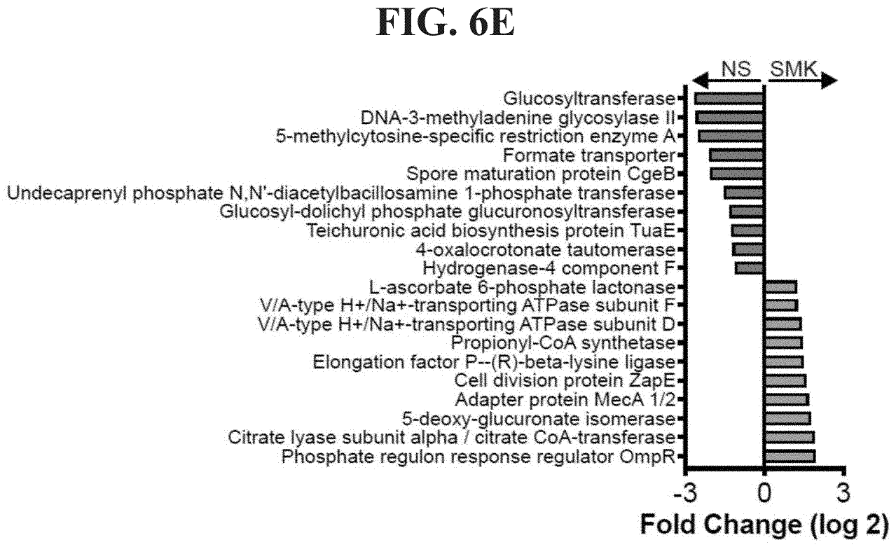

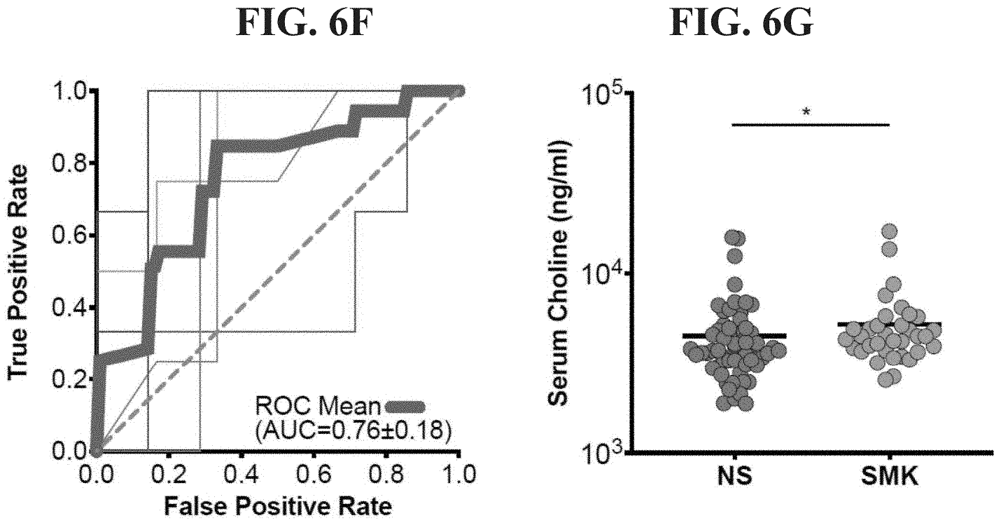

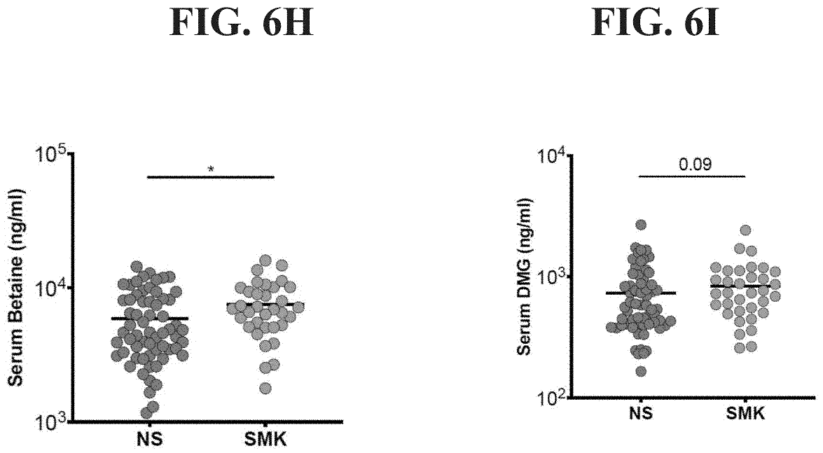

ANOVA and Sidak correction. iAUC (C) describing the weight change at active smoking or cessation of FIG. 4B. One-way ANOVA and Sidak correction. (D) Weight change during active smoking and cessation. NS (n=10), SMK (n=10), NS+abx (n=10), SMK+abx (n=10), SMK+abx+DMG (n=10) mice; Cessation: Day 42: One-way ANOVA and Sidak correction. Inset: iAUC describing the weight change at active smoking or cessation. One-way ANOVA and Sidak corretion. (E) Weight change during active smoking and cessation. NS (n=10), SMK (n=9-10), NS+abx (n=10), SMK+abx (n=10), SMK+abx+PBS (n=10), SMK+abx+DMG (n=10) mice; Cessation: Day 49: One-way ANOVA and Sidak correction. Inset: iAUC describing the weight change at active smoking or cessation. One-way ANOVA and Sidak correction. (F) Serum DMG level assessed by targeted mass spectrometry, in mice consuming Choline-deficient diet (CDD). NS (n=10), SMK (n=10), NS+abx (n=9), SMK+abx (n=10) mice; One-way ANOVA and Sidak correction. (G-H) Weight change during active smoking and cessation in mice consuming CDD. NS (n=10), SMK (n=10), NS+abx (n=9-10), SMK+abx (n=9-10) mice; Cessation: linear mixed model-p=0.001, 3way-ANOVA p=0.002. Final experimental day: One-way ANOVA and Sidak correction. Inset: iAUC describing the weight change at active smoking or cessation. One-way ANOVA and Sidak correction. (I) Pearson correlation of genes from liver, epididymal adipose tissue (EAT) and gut (jejunum), assessed by RNA-seq. The data calculated for 1og2 fold change of genes that were significantly varied between SMK vs. NS mice and SMK+abx vs. NS+abx mice, as well as between non-smoking DMG- and PBS- supplemented mice. Gray background in graphs denotes the cessation period. Symbols or horizontal lines represent the mean, error bars SEM or 10-90 percentiles. *p<0.05; **p<0.01; ***p<0.001; ****p<0.0001.

FIGS. 5A-I. Metabolic consequences of N-formylanthranilic acid, Trigonelline, and N-acetylglycine supplementation to mice (A) Weight change during active smoking and cessation with addition of N-formylanthranilic acid (N-FAN acid). NS+abx (n=8-10), SMK+abx (n=8-10), l SMK+abx+N-FAN acid (N-formylanthranilic acid, n=9) mice; Cessation: Day 49: One-way ANOVA and Tukey correction. Inset: iAUC describing the weight change at active smoking or cessation. One-way ANOVA and Tukey correction. (B) Weight change during active smoking and cessation with addition of trigonelline. NS+abx+PBS (n=10), SMK+abx+PBS (n=9), NS+abx+TRI (n=10), SMK+abx+TRI (n=10) mice; Cessation: 3way-ANOVA p=0.08. Day 35: One-way ANOVA and Sidak correction. Inset: iAUC describing the weight change at active smoking or cessation. One-way ANOVA and Sidak correction. (C) Serum levels of N-acetylglycine during active smoking (day 15) and cessation (day 30). NS (n=5-7), SMK (n=5-6), NS+abx (n=6-8), SMK+abx (n=6-7) mice; 2-way ANOVA and BH correction (q<0.1). (D) Stool levels of N-acetylglycine in naive mice, assessed by untargeted mass spectrometry. SPF (n=6), GF (n=7) mice; Mann Whitney U-test and BH correction. (E) Calories measured in rodent diet. NC (n=3), HFD (n=3), ACG-HFD (n=3); One-way ANOVA and Tukey correction. (F-H) Weight change under HFD or ACG-HFD consumption. HFD (n=5-10-15), ACG-HFD (n=5-10-14) mice; Final experimental day: Unpaired t-test. Inset: iAUC describing the weight change at active smoking or cessation. Unpaired t-test. (I) Weight change under HFD or ACG-HFD consumption at an earlier age (7-weeks-old). HFD (n=19), ACG-HFD (n=20) mice; Day 35: Unpaired t-test. Inset: iAUC describing the weight change at active smoking or cessation. Unpaired t-test. Gray background in graphs denotes the cessation period. Symbols or horizontal lines represent the mean, error bars SEM or 10-90 percentiles. *p<0.05; **p<0.01; ***p<0.001; ****p<0.0001.

FIGS. 6A-I. Potential associations with human smoking (A) Experimental outline of the human cohort. (B-F) Results for analysis of human fecal microbiome (NS n=40, SMK n=20). (B) PCA of metagenomically-assembled genomes (MAGs) relative abundances in human stool; inset: PERMANOVA. (C) Differential abundance results of all MAGs; asterisks denote significant differences (p<0.05); two-sided Mann-Whitney U-test. (D) PCA of KO annotated reads; inset: PERMANOVA. (E) KEGG orthologs of highest effect (feature with negative values enriched in SMK), two-sided Mann-Whitney U-test, highest (and lowest), 2-fold log change. (G) ROC curves for binary classifier (methods). (G-I) Targeted mass spectrometry of metabolites from the DMG biosynthesis pathway: choline (G), betaine (H) and DMG (I); NS (n=62), SMK (n=34); two-sided Mann-Whitney U-test, mean values are presented. *p<0.05; **p<0.01.

### DESCRIPTION OF SPECIFIC EMBODIMENTS OF THE INVENTION

The present invention, in some embodiments thereof, relates to methods of modulating weight and, more particularly, but not exclusively, to reduction of weight following smoking cessation.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

An adverse effect of smoking cessation is weight gain (SCWG). The present inventors, utilizing a multiomic approach have now demonstrated that exposure to cigarette smoke in mice induces pronounced alterations in gut microbial composition and function, mediated in part by the influx of smoking-related compounds into the gastrointestinal tract lumen.

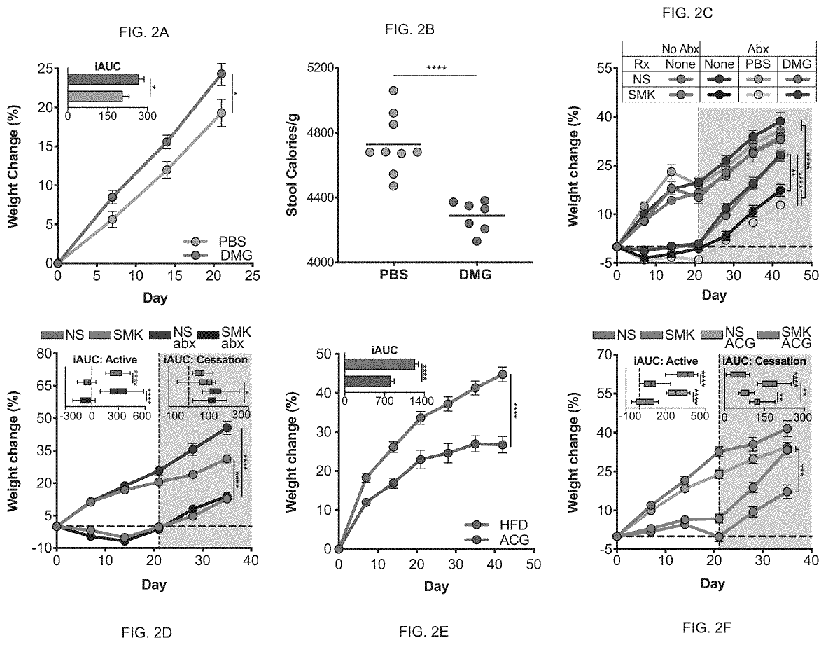

The present inventors have demonstrated that during active smoking, weight lowering effects are dominated by microbiome-independent, smoking-induced weight reduction. However, upon smoking cessation, a distinct dysbiotic microbiome configuration induces a pronounced weight gain, which is abrogated upon antibiotic treatment. This weight gaining phenotype is transferable to high fat diet (HFD)-consuming, non-smoking germ-free (GF) recipient mice by fecal microbiome transplantation (FMT). A combined genomic-metabolomic analysis suggests that several microbiome-modulated metabolites drive SCWG. The first, dimethylglycine (DMG), features an enhanced synthesis from dietary choline during smoking, driven by concerted alteration in host and microbiome biosynthetic pathways. Elevated DMG levels during cessation, in turn, contributed to SCWG through induction of enhanced gastrointestinal energy harvest. As such, DMG supplementation to antibiotics-treated smoking-cessation mice restored SCWG, while administration of a choline-deficient diet, depleting the pathway's main ligand, prevented SCWG in smoking mice (FIG. 2D and FIGS. 4G-H). In contrast, Acetyl-Glycine (ACG) features an opposite, microbiome-modulated weight reduction bioactivity, but is depleted during smoking and smoking-cessation, thereby contributing to SCWG (FIG. 2E, FIGS. 5F-I).

Consequently, the present teachings suggest that regulation of microbiome metabolites may be a promising therapy for prevention of weight gain in general and following nicotine smoking cessation, in particular.

Thus, according to a first aspect of the present invention, there is provided a method of treating obesity in a subject in need thereof, the method comprising administering to the subject a therapeutically effective amount of an agent that specifically increases the amount of hexadecadienoate (16:2n6), N-acetylglycine, 1-palmitoyl-2-gamma-linolenoyl-GPC (16:0/18:3n6) and/or Hexanoylglycine or an agent that decreases the amount of dimethylglycine (DMG) in the fecal metabolome of the subject, thereby treating the obesity.

According to one embodiment the obese subject has a body mass index (BMI) of greater than 30. Subjects having BMI between 25 and 30 are considered overweight and in one embodiment, are treated by the agents disclosed herein. The body mass index (BMI) is calculated by dividing an individual's weight in kilograms by the square of their height in meters. BMI does not distinguish fat mass from lean mass and an obese subject typically has excess adipose tissue.

In one embodiment, the subject has a BMI of 25 or over, e.g. 26, 27, 28, 29, 30, 31, 32, 33, 34, 35, 36, 37, 38, 39 or 40 or greater and has no obesity-related co-morbidity. In one embodiment, the patient is morbidly obese and has a BMI of 40 or over. In one embodiment, the subject is obese and/or suffering from complications associated with obesity. In one embodiment, the subject is obese and/or was suffering from complications associated with obesity, which have now been corrected. In one embodiment, the subject has a Body Mass Index (BMI) of over 25, and preferably over 30.

The agent may be capable of increasing a single metabolite or may be capable of increasing a group of metabolites (e.g. metabolites of a particular metabolic pathway in which the below disclosed metabolites take part).

In one embodiment, the agent is hexadecadienoate (16:2n6), N-acetylglycine, 1-palmitoyl-2-gamma-linolenoyl-GPC (16:0/18:3n6) and/or Hexanoylglycine.

According to a particular embodiment, the agent comprises N-acetylglycine. According to another embodiment, the agent is a combination of at least two of the above disclosed metabolites.

According to another embodiment, the agent is a combination of each of the three of the above disclosed metabolites.

According to still another embodiment, the agent comprises no more than 20 metabolites that are found in the human fecal metabolome.

According to still another embodiment, the agent comprises no more than 20 metabolites that are found in the human serum metabolome.

According to still another embodiment, the agent comprises no more than 10 metabolites that are found in the human fecal metabolome.

According to still another embodiment, the agent comprises no more than 10 metabolites that are found in the human serum metabolome.

As used herein, a “metabolite” is an intermediate or product of metabolism. The term metabolite is generally restricted to small molecules and does not include polymeric compounds such as DNA or proteins. A metabolite may serve as a substrate for an enzyme of a metabolic pathway, an intermediate of such a pathway or the product obtained by the metabolic pathway.

As used herein, the term “metabolome” refers to the chemical profile or fingerprint of the metabolites in a bodily fluid, feces, a cell, a tissue, an organ, or an organism.

In combination with (or instead of) at least one of the agents that increases the amount of hexadecadienoate (16:2n6), N-acetylglycine, 1-palmitoyl-2-gamma-linolenoyl-GPC (16:0/18:3n6) and Hexanoylglycine, the present inventors further contemplate agents that decrease the amount of dimethylglycine (DMG) in the fecal metabolome of the subject.

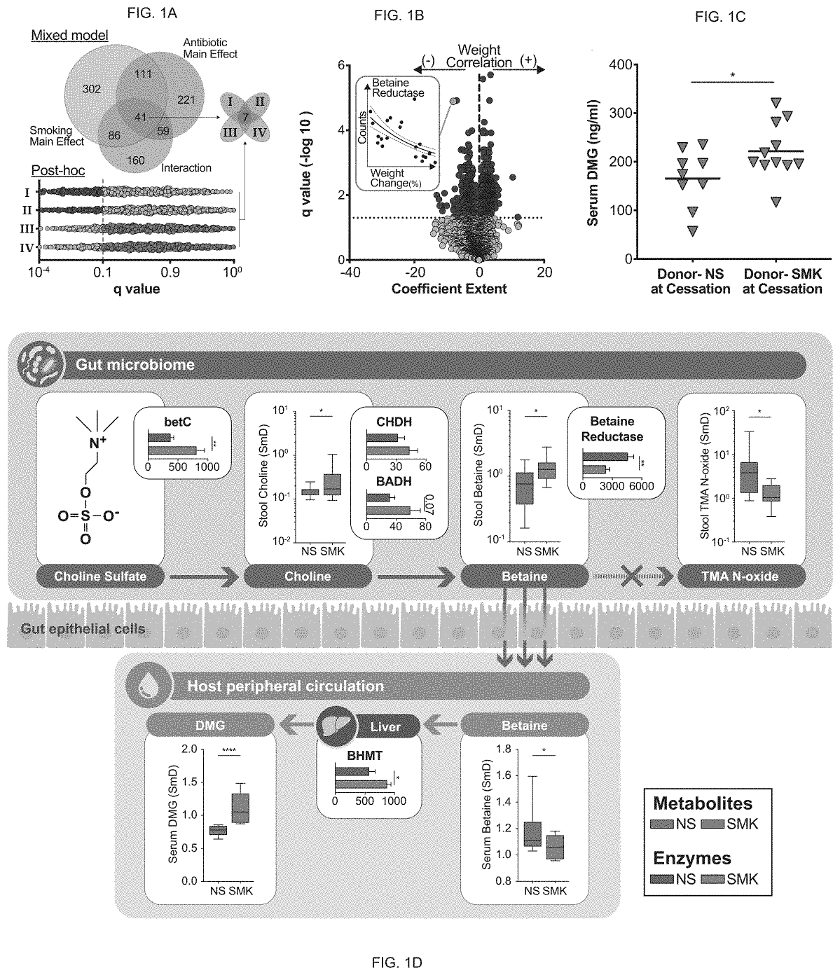

Such agents include inhibitors of the DMG synthesis pathway (as illustrated in FIG. 1D), inhibitors of DMG signaling or a choline-poor diet.

Thus, in one embodiment, the agent may be an inhibitor of choline dehydrogenase in the gut microbiome of the subject. In another embodiment, the agent may enhance the activity of betaine reductase in the gut microbiome of the subject, thereby reducing the amount of betaine in the host peripheral circulation. In still another embodiment, the agent may reduce the amount and/or activity of betaine-homocysteine methyltransferase.

The present inventors further propose it is possible to reduce the risk of weight gain in a subject who has quit smoking by administering to the subject agents that modulate his/her fecal metabolome in a way such that his/her fecal metabolome becomes more similar to that of a healthy, non-obese, non-smoking subject.

The subject who has quit smoking, typically has smoked at least 1 cigarette a day, at least 2 cigarettes a day, at least 3 cigarettes a day, at least 4 cigarettes a day, at least 5 cigarettes a day, at least 6 cigarettes a day, at least 8 cigarettes a day, at least 9cigarettes a day, at least 10 cigarettes a day, at least 1 packet a day or more.

According to this aspect of the present invention the subject is not obese - for example has a BMI lower than 25.

In another embodiment, the subject has a BMI of 25 or over, e.g. 26, 27, 28, 29, 30, 31, 32, 33, 34, 35, 36, 37, 38, 39 or 40 or greater and has no obesity-related co-morbidity.

One method of altering the fecal metabolome of the subject who has quit smoking is by administering microbes from a fecal microbiome of a non-smoker (preferably, a healthy, non-obese non-smoker).

The microbial compositions of this aspect of the present invention may be statistically significantly similar to a microbiome of a non-obese subject who has not smoked (e.g. in the last year, preferably last two years).

The microbial compositions may be taken from a microbiota sample (e.g. feces) of a non-obese, healthy, non-smoker.

A microbiota sample comprises a sample of microbes and or components or products thereof from a microbiome.

In one embodiment, the agent is a fecal transplant.

In some embodiments, the fecal transplant is processed fecal material (fecal filtrate) having reduced volume and/or fecal aroma relative to unprocessed fecal material. In certain embodiments, the fecal transplant is a fecal bacterial sample. The term fecal transplant may also be used to refer to the process of transplantation of fecal bacteria isolated from the non-smoker into a recipient. The process may be also referred to as fecal microbiota transplantation (FMT), stool transplant or bacteriotherapy.

In one embodiment, the fecal donor has no risk factors for transmissible diseases and has not been exposed to agents, such as, for example, antibiotics, that could alter the composition of their gut microbiota. Fecal transplant donor selection criteria and screening tests are outlined in detail in published international guidelines established by the FMT Working Group (Bakken et al. Clin Gastroenterol Hepatol. 9:1044-9, 2011). Details pertaining to the harvesting and processing of fecal transplant material are known in the art and are reviewed in Borody et al. (Curr Gastroenterol Rep. 15: 337, 2013). Briefly, many protocols call for use of fresh feces, which requires collection and processing on the same day scheduled for the FMT. Other protocols have been developed that use highly filtered human microbiota mixed with a cryoprotectant, which can be frozen for storage at -80.degree. C. until required for use (Hamilton et al. Am J Gastroenterol. 107(5):761-7, 2012). This approach benefits from convenience with regard to scheduling, and generates a processed fecal material (fecal filtrate) having reduced volume and fecal aroma. Equivalent clinical efficacy can be expected when either purified processed fecal material or fresh, partly filtered feces are used in the disclosed methods. In some embodiments, the microorganism in the transplant can have undergone processing in order for it to increase its survival.

It is not uncommon to find unknown bacteria, unculturable bacteria, or mixed cultures of bacteria in a fecal sample. In some embodiments, the composition can include one or more unknown and/or unculturable bacteria. The term “unculturable” as used herein refers to a given bacterium that current laboratory culturing techniques are unable to grow in the laboratory. An unculturable bacterium does not mean “a bacterium that can never be cultured” but, rather, signifies the lack of critical information on their biology. In some embodiments, the compositions described herein can include a substantially unculturable bacterium. The term “substantially unculturable” refers to a strain that, when cultured under normal laboratory conditions, less than 20% of replicates of that strain will reach a logarithmic growth phase, for example less than 20%, 15%, 10%, 5%, 2%, 1%, or 0.1%. Unknown and unculturable bacteria can be placed in taxonomic groups by amplifying their 16S rRNA gene, and subsequently their signature amplicon pattern can be recognized if they are encountered again.

Alternatively, the microbial composition may be artificially created by adding known amounts of different microbes.

It will be appreciated that the microbial composition which is derived from the microbiota sample of the non-smoking subject may be manipulated prior to administrating by increasing the amount of a particular strain or depleting the amount of a particular strain. Alternatively, the microbial compositions are treated in such a way so as not to alter the relative balance between the microbial species and taxa comprised therein.

In some embodiments, the microbial composition is expanded ex vivo using known culturing methods prior to administration. In other embodiments, the microbial composition is not expanded ex vivo prior to administration.

According to one embodiment, the microbial composition is not derived from fecal material.

According to still another embodiment, the microbial composition is devoid (or comprises only trace quantities) of fecal material (e.g, fiber).

The microbial composition may be in any suitable form, for example in a powdered dry form. In addition, the microorganism/s of the composition may have undergone processing in order for it to increase its/their survival. For example, the microorganism may be coated or encapsulated in a polysaccharide, fat, starch, protein or in a sugar matrix. Standard encapsulation techniques known in the art can be used. For example, techniques discussed in U.S. Pat. No. 6,190,591, which is hereby incorporated by reference in its entirety, may be used.

According to a particular embodiment, the microbial composition is formulated in a food product, functional food or nutraceutical.

In some embodiments, a food product, functional food or nutraceutical is or comprises a dairy product. In some embodiments, a dairy product is or comprises a yogurt product. In some embodiments, a dairy product is or comprises a milk product.

In some embodiments, a dairy product is or comprises a cheese product. In some embodiments, a food product, functional food or nutraceutical is or comprises a juice or other product derived from fruit. In some embodiments, a food product, functional food or nutraceutical is or comprises a product derived from vegetables. In some embodiments, a food product, functional food or nutraceutical is or comprises a grain product, including but not limited to cereal, crackers, bread, and/or oatmeal. In some embodiments, a food product, functional food or nutraceutical is or comprises a rice product. In some embodiments, a food product, functional food or nutraceutical is or comprises a meat product.

Another method of altering the fecal metabolome of the subject who has quit smoking is by administering at least one metabolite from a fecal metabolome of a non-smoker (preferably, a healthy, non-obese non-smoker).

Thus, according to another aspect of the present invention there is provided a method of reducing the risk of weight gain following nicotine smoking cessation in a subject comprising administering to the subject an effective amount of an agent that specifically increases the amount of hexadecadienoate (16:2n6), N-acetylglycine, 1-palmitoyl-2-gamma-linolenoyl-GPC (16:0/18:3n6) and/or Hexanoylglycine; or an agent that decreases the amount of dimethylglycine (DMG) in the fecal metabolome of the subject, thereby reducing the risk of weight gain in the subject.

Exemplary agents that may be provided include the metabolites themselves - namely hexadecadienoate (16:2n6), N-acetylglycine, 1-palmitoyl-2-gamma-linolenoyl-GPC (16:0/18:3n6) and Hexanoylglycine.

According to a particular embodiment, the agent comprises at least one of the above disclosed metabolites e.g. N-acetylglycine.

Furthermore, the present invention contemplates agents that decreases the amount of dimethylglycine (DMG) for preventing weight gain in the subject who has quit smoking. Such agents are described herein above.

The agents of this aspect of the present invention are typically provided immediately following smoking cessation, e.g. at least 12 hours following smoking cessation, 24 hours following smoking cessation or even 48 hours following smoking cessation. Preferably, the treatment starts no later than 1 month following smoking cessation. Preferably, the treatment starts no later than 1 week following smoking cessation.

The metabolites (or agents that directly increase or decrease the above disclosed metabolites) may be formulated into food products as further described herein below.

In one embodiment, at least one of the above disclosed metabolites (e.g. N-acetylglycine) is formulated into a chewing gum.

Chewing gums may comprise a chewing gum base comprising elastomers, e.g. polyvinyl acetate (PVA), polyethylene, (low or medium molecular) polyiso butane (PIB), polybutadiene, isobutene/isoprene copolymers, polyvinyl ethyl ether (PVE), polyvinyl butyl ether, copolymers of vinyl esters and vinyl ethers, styrene/butadiene copolymers (SBR) or vinyl elastomers, e.g. based on vinyl acetate/vinyl laurate, vinyl acetate/vinyl stearate or ethylene/vinyl acetate and mixtures of the mentioned elastomers as e.g. example described EP 0 242 325, U.S. Pat. Nos. 4,518,615, 5,093,136, 5,266,336 5,601,858 or 6,986,709. Additionally chewing gum bases may contain further ingredients, e.g. (mineral) filers, e.g. calcium carbonate, titanium dioxide, silicone dioxide, talcum, aluminum oxide, dicalcium phosphate, tricalcium phosphate, magnesium hydroxide and mixtures thereof, plasticisers (e.g. lanolin, stearic acid, sodium stearate, ethyl acetate, diacetin (glycerol diacetate), triacetin (glycerol triacetate) and trietyhl citrate), emulsifiers (e.g. phosphatides, such as lecithin and mono and diglycerides of fatty acids, e.g. glycerol monostearate), antioxidants, waxes (e.g. paraffine waxes, candelilla waxes, carnauba waxes, microcrystalline waxes and polyethylene waxes), fats or fatty oils (e.g. hardened (hydrogenated) plant or animal fats) and mono, di or triglycerides.

The chewing gum can further comprise nicotine.

The bacterial metabolite may be provided per se or as part of a pharmaceutical composition, where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to one or more of the bacterial metabolites described herein accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, inrtaperitoneal, intranasal, or intraocular injections.

According to a particular embodiment, the agent is administered orally or rectally.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (e.g. nicotinamide) effective to prevent, alleviate or ameliorate symptoms of a disorder (e.g., ALS) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide blood, brain or CSF levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

The metabolites of the present invention may be provided in a food (such as food bars, biscuits, snack foods and other standard food forms well known in the art), or in drink formulations. Drinks can contain flavoring, buffers and the like. Nutritional supplements comprising the metabolites of the present invention are also contemplated.

Prior to administration of the probiotic or metabolite, the subject may be pretreated with an agent which reduces the number of naturally occurring microbes in the microbiome (e.g. by antibiotic treatment). According to a particular embodiment, the treatment significantly eliminates the naturally occurring gut microflora by at least 20%, 30% 40%, 50%, 60%, 70%, 80% or even 90%.

Thus, the present inventors contemplate the use of antibiotic agents for preventing weight gain after smoking cessation.

In addition, the use of antibiotic agents may be recommended for preventing weight gain after smoking cessation, irrespective of whether a probiotic or metabolite is administered.

As used herein, the term “antibiotic agent” refers to a group of chemical substances, isolated from natural sources or derived from antibiotic agents isolated from natural sources, having a capacity to inhibit growth of, or to destroy bacteria. Examples of antibiotic agents include, but are not limited to; Amikacin; Amoxicillin; Ampicillin; Azithromycin; Azlocillin; Aztreonam; Aztreonam; Carbenicillin; Cefaclor; Cefepime; Cefetamet; Cefinetazole; Cefixime; Cefonicid; Cefoperazone; Cefotaxime; Cefotetan; Cefoxitin; Cefpodoxime; Cefprozil; Cefsulodin; Ceftazidime; Ceftizoxime; Ceftriaxone; Cefuroxime; Cephalexin; Cephalothin; Cethromycin; Chloramphenicol; Cinoxacin; Ciprofloxacin; Clarithromycin; Clindamycin; Cloxacillin; Co-amoxiclavuanate; Dalbavancin; Daptomycin; Dicloxacillin; Doxycycline; Enoxacin; Erythromycin estolate; Erythromycin ethyl succinate; Erythromycin glucoheptonate; Erythromycin lactobionate; Erythromycin stearate; Erythromycin; Fidaxomicin; Fleroxacin; Gentamicin; Imipenem; Kanamycin; Lomefloxacin; Loracarbef; Methicillin; Metronidazole; Mezlocillin; Minocycline; Mupirocin; Nafcillin; Nalidixic acid; Netilmicin; Nitrofurantoin; Norfloxacin; Ofloxacin; Oxacillin; Penicillin G; Piperacillin; Retapamulin; Rifaxamin, Rifampin; Roxithromycin; Streptomycin; Sulfamethoxazole; Teicoplanin; Tetracycline; Ticarcillin; Tigecycline; Tobramycin; Trimethoprim; Vancomycin; combinations of Piperacillin and Tazobactam; and their various salts, acids, bases, and other derivatives. Anti-bacterial antibiotic agents include, but are not limited to, aminoglycosides, carbacephems, carbapenems, cephalosporins, cephamycins, fluoroquinolones, glycopeptides, lincosamides, macrolides, monobactams, penicillins, quinolones, sulfonamides, and tetracyclines.

In one embodiment, the antibiotic is a broad spectrum antibiotic (e.g. vancomycin, neomycin, ampicillin, and metronidazole).

In another embodiment, the antibiotic is a narrow spectrum antibiotic.

Antibacterial agents also include antibacterial peptides. Examples include but are not limited to abaecin; andropin; apidaecins; bombinin; brevinins; buforin II; CAP18; cecropins; ceratotoxin; defensins; dermaseptin; dermcidin; drosomycin; esculentins; indolicidin; LL37; magainin; maximum H5; melittin; moricin; prophenin; protegrin; and or tachyplesins.

It will be appreciated that the above described method for reducing the risk or weight gain in a subject that has quit smoking may be effected in a personalized or non-personalized fashion.

As a personalized therapy, the method includes a step of determining the amount of hexadecadienoate (16:2n6), N-acetylglycine, 1-palmitoyl-2-gamma-linolenoyl-GPC (16:0/18:3n6), Hexanoylglycine and DMG in a fecal sample of the subject wherein when the level of DMG is above a predetermined amount and/or the level of hexadecadienoate (16:2n6), N-acetylglycine, 1-palmitoyl-2-gamma-linolenoyl-GPC (16:0/18:3n6) or Hexanoylglycine is below a predetermined amount, it is indicative that the subject has a predisposition to weight gain on cessation of nicotine smoking.

According to a specific embodiment, when the level of DMG is at least 2 fold, 5 fold or 10 fold higher than the amount of DMG found in a fecal sample of a healthy non-smoker, then it is indicative that the subject has a predisposition to weight gain on cessation of nicotine smoking.

According to a specific embodiment, when the level of hexadecadienoate (16:2n6) is at least 2 fold, 5 fold or 10 fold lower than the amount of hexadecadienoate (16:2n6) found in a fecal sample of a healthy non-smoker, then it is indicative that the subject has a predisposition to weight gain on cessation of nicotine smoking.

According to a specific embodiment, when the level of N-acetylglycine is at least 2 fold, 5 fold or 10 fold lower than the amount of N-acetylglycine found in a fecal sample of a healthy non-smoker, then it is indicative that the subject has a predisposition to weight gain on cessation of nicotine smoking.

According to a specific embodiment, when the level of 1-palmitoyl-2-gamma-linolenoyl-GPC (16:0/18:3n6) is at least 2 fold, 5 fold or 10 fold lower than the amount of 1-palmitoyl-2-gamma-linolenoyl-GPC (16:0/18:3n6) found in a fecal sample of a healthy non-smoker, then it is indicative that the subject has a predisposition to weight gain on cessation of nicotine smoking.

According to a specific embodiment, when the level of Hexanoylglycine is at least 2 fold, 5 fold or 10 fold lower than the amount of Hexanoylglycine found in a fecal sample of a healthy non-smoker, then it is indicative that the subject has a predisposition to weight gain on cessation of nicotine smoking.

The fecal sample may be frozen and/or lyophilized prior to analysis. According to another embodiment, the sample may be subjected to solid phase extraction methods.

### Quantifying Metabolite Levels:

In one embodiment, metabolites are identified using a physical separation method.

The term “physical separation method” as used herein refers to any method known to those with skill in the art sufficient to produce a profile of changes and differences in small molecules produced in hSLCs, contacted with a toxic, teratogenic or test chemical compound according to the methods of this invention. In a preferred embodiment, physical separation methods permit detection of cellular metabolites including but not limited to sugars, organic acids, amino acids, fatty acids, hormones, vitamins, and oligopeptides, as well as ionic fragments thereof and low molecular weight compounds (preferably with a molecular weight less than 3000 Daltons, and more particularly between 50 and 3000 Daltons). For example, mass spectrometry can be used. In particular embodiments, this analysis is performed by liquid chromatography/electrospray ionization time of flight mass spectrometry (LC/ESI-TOF-MS), however it will be understood that metabolites as set forth herein can be detected using alternative spectrometry methods or other methods known in the art for analyzing these types of compounds in this size range.

Certain metabolites can be identified by, for example, gene expression analysis, including real-time PCR, RT-PCR, Northern analysis, and in situ hybridization.

In addition, biomarkers can be identified using Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, tandem mass spectrometry (e.g., MS/MS, MS/MS/MS, ESI-MS/MS etc.), secondary ion mass spectrometry (SIMS), or ion mobility spectrometry (e.g. GC-IMS, IMS-MS, LC-IMS, LC-IMS-MS etc.).

Mass spectrometry methods are well known in the art and have been used to quantify and/or identify biomolecules, such as proteins and other cellular metabolites (see, e.g., Li et al., 2000; Rowley et al., 2000; and Kuster and Mann, 1998).

In certain embodiments, a gas phase ion spectrophotometer is used. In other embodiments, laser-desorption/ionization mass spectrometry is used to identify metabolites. Modern laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”).

In MALDI, the metabolite is mixed with a solution containing a matrix, and a drop of the liquid is placed on the surface of a substrate. The matrix solution then co-crystallizes with the biomarkers. The substrate is inserted into the mass spectrometer. Laser energy is directed to the substrate surface where it desorbs and ionizes the proteins without significantly fragmenting them.

However, MALDI has limitations as an analytical tool. It does not provide means for fractionating the biological fluid, and the matrix material can interfere with detection, especially for low molecular weight analytes.

In SELDI, the substrate surface is modified so that it is an active participant in the desorption process. In one variant, the surface is derivatized with adsorbent and/or capture reagents that selectively bind the biomarker of interest. In another variant, the surface is derivatized with energy absorbing molecules that are not desorbed when struck with the laser. In another variant, the surface is derivatized with molecules that bind the biomarker of interest and that contain a photolytic bond that is broken upon application of the laser. In each of these methods, the derivatizing agent generally is localized to a specific location on the substrate surface where the sample is applied. The two methods can be combined by, for example, using a SELDI affinity surface to capture an analyte (e.g. biomarker) and adding matrix-containing liquid to the captured analyte to provide the energy absorbing material.

For additional information regarding mass spectrometers, see, e.g., Principles of Instrumental Analysis, 3rd edition., Skoog, Saunders College Publishing, Philadelphia, 1985; and Kirk-Othmer Encyclopedia of Chemical Technology, 4.sup.th ed. Vol. 15 (John Wiley & Sons, New York 1995), pp. 1071-1094.

In some embodiments, the data from mass spectrometry is represented as a mass chromatogram. A “mass chromatogram” is a representation of mass spectrometry data as a chromatogram, where the x-axis represents time and the y-axis represents signal intensity. In one aspect the mass chromatogram is a total ion current (TIC) chromatogram. In another aspect, the mass chromatogram is a base peak chromatogram. In other embodiments, the mass chromatogram is a selected ion monitoring (SIM) chromatogram. In yet another embodiment, the mass chromatogram is a selected reaction monitoring (SRM) chromatogram. In one embodiment, the mass chromatogram is an extracted ion chromatogram (EIC).

In an EIC, a single feature is monitored throughout the entire run. The total intensity or base peak intensity within a mass tolerance window around a particular analyte's mass-to-charge ratio is plotted at every point in the analysis. The size of the mass tolerance window typically depends on the mass accuracy and mass resolution of the instrument collecting the data. As used herein, the term “feature” refers to a single small metabolite, or a fragment of a metabolite. In some embodiments, the term feature may also include noise upon further investigation.

Detection of the presence of a metabolite will typically involve detection of signal intensity. This, in turn, can reflect the quantity and character of a biomarker bound to the substrate. For example, in certain embodiments, the signal strength of peak values from spectra of a first sample and a second sample can be compared (e.g., visually, by computer analysis etc.) to determine the relative amounts of particular metabolites. Software programs such as the Biomarker Wizard program (Ciphergen Biosystems, Inc., Fremont, Calif.) can be used to aid in analyzing mass spectra. The mass spectrometers and their techniques are well known.

A person skilled in the art understands that any of the components of a mass spectrometer, e.g., desorption source, mass analyzer, detect, etc., and varied sample preparations can be combined with other suitable components or preparations described herein, or to those known in the art. For example, in some embodiments a control sample may contain heavy atoms, e.g. ¹³C, thereby permitting the test sample to be mixed with the known control sample in the same mass spectrometry run. Good stable isotopic labeling is included.

In one embodiment, a laser desorption time-of-flight (TOF) mass spectrometer is used. In laser desorption mass spectrometry, a substrate with a bound marker is introduced into an inlet system. The marker is desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of molecules of specific mass to charge ratio.

In one embodiment of the invention, levels of metabolites are detected by MALDI-TOF mass spectrometry.

Methods of detecting metabolites also include the use of surface plasmon resonance (SPR). The SPR biosensing technology has been combined with MALDI-TOF mass spectrometry for the desorption and identification of metabolites.

Data for statistical analysis can be extracted from chromatograms (spectra of mass signals) using softwares for statistical methods known in the art. “Statistics” is the science of making effective use of numerical data relating to groups of individuals or experiments. Methods for statistical analysis are well-known in the art.

In one embodiment a computer is used for statistical analysis.

In one embodiment, the Agilent MassProfiler or MassProfilerProfessional software is used for statistical analysis. In another embodiment, the Agilent MassHunter software Qual software is used for statistical analysis. In other embodiments, alternative statistical analysis methods can be used. Such other statistical methods include the Analysis of Variance (ANOVA) test, Chi-square test, Correlation test, Factor analysis test, Mann-Whitney U test, Mean square weighted derivation (MSWD), Pearson product-moment correlation coefficient, Regression analysis, Spearman's rank correlation coefficient, Student's T test, Welch's T-test, Tukey's test, and Time series analysis.

In different embodiments, signals from mass spectrometry can be transformed in different ways to improve the performance of the method. Either individual signals or summaries of the distributions of signals (such as mean, median or variance) can be so transformed. Possible transformations include taking the logarithm, taking some positive or negative power, for example the square root or inverse, or taking the arcsin (Myers, Classical and Modern Regression with Applications, 2nd edition, Duxbury Press, 1990).

If the subject has been found to have a predisposition to weight gain on cessation of nicotine smoking, the subject may be recommended to enter a weight loss program or exercise regime.

Non-limiting examples of dietary weight loss programs include but are not limited to a South Beach Diet, a Dukin diet, a Stillman diet, an Atkins Diet, a gluten-free diet, a ketogenic diet, a low-residue diet, a liquid diet, a vegetarian diet, a low-calorie diet (e.g., Weight Watches, Jenny Craig, Nutrisystems), a low-fat diet, a low-carbohydrate diet, a low-protein diet, a low-monosodium glutamate (MSG) diet, a detox diet, an elimination diet, a specific carbohydrate diet, a diabetic diet, a dietary approach to stop hypertension diet (DASH) diet, a best bet diet, an organic diet, and combinations thereof.

Since increased levels of DMG have been shown to be associated with weight gain, the present inventors also contemplate administration of DMG for treating diseases associated with weight loss. Conversely, since decreased levels of hexadecadienoate (16:2n6), N-acetylglycine, 1-palmitoyl-2-gamma-linolenoyl-GPC (16:0/18:3n6) and Hexanoylglycine have been shown to be associated with weight gain, the present inventors also contemplate administration of agents that decrease said metabolites for treating diseases associated with weight loss.

Agents that decrease hexadecadienoate (16:2n6), N-acetylglycine, 1-palmitoyl-2-gamma-linolenoyl-GPC (16:0/18:3n6) and/or Hexanoylglycine, include inhibitors of their synthesis pathway, inhibitors of their signaling or diets which are low in same.

Examples of diseases associated with weight loss include, but are not limited to cancer, chemotherapy induced weight loss, hyperthyroidism, cathexia and anorexia.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells - A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization- A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### MATERIALS AND METHODS

Mice. Eight-weeks old C57BL/6J male mice were purchased from Harlan. All mice were maintained in the animal facility of the Weizmann Institute and acclimatized for two weeks before the initiation of experiments. Mice were fed on normal chow (NC) or switched to a high-fat diet (HFD, D12492, 60% kcal from fat, Research Diets)/ choline-deficient diet (CDD, rodent diet with 60 kcal % fat without added Choline) three days prior to the experimental starting point. Only in experiments using ACG (HFD containing 60 kcal % fat and 7500 mg N-Acetylglycine/kg Diet), mice were fed HFD instead of NC, which was replaced with ACG three days prior to cessation. For all experiments, the mice were assigned to each group based on their weight in order to create weight-matched groups and eliminate weight differences at the beginning of the experiments. Two weeks before experimental starting point, antibiotic-treated groups were given a combination of vancomycin (0.5 g/l), ampicillin (1 g/l), neomycin (1 g/l), and metronidazole (1 g/l) ad libitum in their drinking water, referred to as 4-way abx. All antibiotics were purchased from Sigma Aldrich. The mice were weighed at least once a week to calculate the percentage weight change induction by HFD. Lean and fat mass was determined by nuclear magnetic resonance (NMR) using Bruker minispec LF50.mq7.5 MHZ live mice analyzer.

Cigarette Smoke Exposure. A smoking machine (model TE-10; Teague Enterprises) was used to generate the mice cigarette smoke exposure model. Mice were exposed to cigarette smoke five days per week for three weeks. Each daily exposure period lasted 144 minutes, with one rest interval of 120 minutes. Smoke-exposed mice were placed in a whole-body exposure chamber attached to the smoke apparatus, mixing chamber, and air pump. Mainstream and side-stream smoke were mixed and allowed to flow through the chamber. Kentucky 3R4F reference cigarettes (Tobacco and Health Research Institute, University of Kentucky) were constantly burning at all times and smoked using the Federal Trade Commission method, which consists of 2-s puffs of 35 cc each at one-minute intervals. Each cigarette was smoked for a duration of nine minutes. The chamber flow rate and the number of cigarettes smoked in parallel were adjusted to reach 150 mg/m³ intermediate levels of smoke inside the whole-body exposure chamber. Smoke levels were measured at least twice daily. The cigarettes were stored at 4° C. until needed. At least 48 hours prior to use, the cigarettes were placed in a closed chamber, along with a solution of glycerin/water (mixed in a ratio of 0.76/0.26) to establish a relative humidity of 60%.

Glucose Tolerance Test. Mice were fasted overnight for 12 hours, with free access to water. Blood from the tail vein was used to measure glucose levels using a glucometer (ContourTM blood glucose meter, Bayer). An intra-peritoneal injection with 2 g/kg-1 of glucose (J. T. Baker) was injected into mice, following glucose measurement at intervals of 15, 30, 60, 90, and 120 min.

Bomb calorimetry. Mice were transferred to a clean single housed cage, fecal samples collected and stored at −80° C. Before use, samples were dried with FreeZone 4.5-liter cascade benchtop freeze dry system (Labconco) for 24 hours. Gross energy content was measured using a water handling system (6510) and a bomb calorimeter (6200) by Parr Instrument Co. The calorimeter energy equivalent factor was determined using benzoic acid standards.

Metabolic Cages. Metabolic cages (PhenoMaster system, TSE-Systems, Bad Homburg, Germany) were used to measured several parameters including-food and liquid intake, energy expenditure and locomotors activity. All parameters were measured continuously and simultaneously. The system consists a combination of sensitive feeding sensors for automated measurements, gas calorimetry to calculate energy expenditure for each mouse, a photobeam-based activity monitoring system for ambulatory movements recording and indirect gas calorimetry to calculate energy expenditure in each cage. The metabolic parameters were measured while mice were placed in a single metabolic-cage. Mice were trained for one week in training cages before data acquisition. In order to reduce the noise caused by mice transfer, we excluded the data acquired in the first 24 hours. The randomly selected mice from smoking experiments were transferred into the metabolic cages immediately after the last smoking exposure.

Blood Samples. Mice were anesthetized with an intraperitoneal (IP) injection of Ketamine (100 mg/kg) and Xylazine (10 mg/kg) mixture. Blood was collected by retro-orbital sinus puncture via the medial canthus of the eye using glass capillaries. Blood samples were collected in heparin-coated tubes and kept on ice. Then, the samples were inserted into pre-chilled centrifuges and spin for 15 min at 10,000 g. The serum was separated and stored in −80° C. until use. Serum biochemistry values (ALT and AST activity, cholesterol levels, ammonia, triglycerides) were obtained using the Roche Cobas 111 Serum analyser according to the manufacturer's instructions.

Fecal Microbiome Transplantation (FMT). Swiss Webster germ-free mice were bred in the Weizmann Institute's germ-free facility and routinely monitored for sterility. For the fecal transplantation experiment, 200 mg stool per recipient (from frozen glycerol donor mouse pellets) was suspended in PBS, vortexed until dissolved and filtered through a 70 μm strainer. Recipient mice received 200 μl of the filtrate solution by oral gavage. After transplantation, all mice were housed in iso-cages in order to maintain sterility.

16S rDNA Sequencing. DNA was isolated from fecal samples stored at −80C using an Invitrogen kit, according to the manufacturer instructions For 16S amplicon pyrosequencing, PCR amplification was performed on the entire V4 region of the 16S rDNA gene, using the 515F (GTGCCAGCMGCCGCGGTAA; SEQ ID NO: 1)/806R (GGACTACHVGGGTWTCTAAT; SEQ ID NO: 2) primer pair. Sequencing of these amplicons was performed on an Illumina MiSeq sequencer, using 2×250 bp paired-end reads.

16s rDNA Analysis. Miseq (Illumina) output files were analyzed using Qiime2 (version 2018.4.0). Using the demux emp-paired command line and the barcode mapping file, the reads were demultiplexed and assigned to their corresponding sample. In the next step, we applied the DADA2 plugin to the reads, using Qiime2 dada2 denoise-paired. In brief the DADA2 pipeline quality filters and trims the reads, pairs the forward and reverse reads and creates an amplicon sequence variant (ASV) table. In order to equalize the sequencing effort among the samples, we plotted alpha rarefaction curves and set an appropriate subsampling depth using diversity alpha-rarefaction. Samples not reaching the established sequencing depths were excluded from further analysis. ASVs were assigned with taxonomic annotations using a naive Bayes fitted classifier pre-trained on murine samples, 99% using the identity Greengenes rRNA database. Relative abundance tables were calculated using Qiime2 feature-classifier classify-sklearn and metadata tabulate. Ordination plots were calculated from Bray-Curtis and Jaccard dissimilarity matrix using principal coordinate analysis (PCoA)¹⁰³ . Functional composition from 16S rDNA performed by PICRUSt2 plugin for Qiime2 (version 2019.7.0)¹⁰⁴⁻¹⁰⁸.

Shotgun Metagenomics Sequencing. For shotgun sequencing, Illumina libraries were prepared using Nextera DNA S amp Prep kit (Illumina, FC-121-1031), according to manufacture protocol and sequenced on the Illumina NextSeq platform with a read length of 80 bp.

Metagenomic Analysis. Bcl2fastq was used to convert the data on the sequencer to fastq files.

Reads were then QC trimmed using Trimmomatic¹⁰⁹, then, bowtie2 search against the mm10 host reference was performed and reads matching the host reference were removed. For SPF experimental setup, we subsampled the read files of all samples to a uniform depth of 11.4 million reads per sample (5.7 per mate). Taxonomic annotation of short reads was performed by Kraken2¹¹⁰ followed by the execution of Bracken¹¹¹. Functional annotations was done using diamond¹¹² via translated search (blastx) of the short reads against reference database including all EC annotated entries of bacterial organisms found in UniProt. For GF experimental setup, a Kegg annotated gene catalog was constructed out of metagenome-assembled genomes (MAGs) which were found using Anvi'o software¹¹³ following the methodology described in ¹¹⁴ In short, a co-assembly of contigs was created for each experimental group using MegaHit¹¹⁵, for each contigs db, HMMER¹¹⁶ was then used to identify bacterial and archeal genes. Taxonomic identification was done using Centrifuge¹¹⁷. Each sample was then mapped to its group's scaffolds using Bowtie2¹¹⁸ and mapping results were profiled by coverage and detection estimation of each scaffold. Profiles of the same group were then merged and contigs therein binned using CONCOCT¹¹⁹. After manual curation of the bins, MAGs were defined as bins with >70% completion in <10% redundancy. Dereplication of the MAGs from all profiles was done using fastANI¹²⁰ (97% similarity and kmer size of 10). Gene calls with Kegg annotations were then extracted from the non-redundant MAGs collection to form the gene catalog. Reads from all samples were mapped to this catalog using Bowtie2. Statistical analysis of counts data analysis was conducted using DESeq2¹²¹.

Human cohort metagenomic analysis. The human data was single-end sequenced and rarefied to 3 M reads per sample. Annotation of the human metagenomes was done using a metagenome-assembled genomes (MAGs) reference catalog, assembled using metagenomes of the murine fecal transplant experiment. A KEGG annotated gene catalog was constructed out of MAGs found using Anvi'o software, following the methodology described earlier (

Delmont, T. O. et al. Nat Microbiol 3, 804-813, doi:10.1038/s41564-018-0176-9 (2018). Specifically, a co-assembly of contigs was created for the union of all reads from each group in the experiment using MegaHit (Li, D et al., Bioinformatics 31, 1674-1676, doi:10.1093/bioinformatics/btv033 (2015). For each contigs db, HMMER (Eddy, S. R. PLoS Comput Biol 7, e1002195, doi:10.1371/journal.pcbi.1002195 (2011)) was then used to identify bacterial and archeal genes. Taxonomic identification was done using Centrifuge (Kim, D., Genome Res 26, 1721-1729, doi:10.1101/gr.210641.116 (2016)). Each sample was then mapped to its group's scaffolds using bowtie2 and mapping results were profiled by coverage and detection estimation of each scaffold. Profiles of the same group were then merged and contigs therein binned using CONCOCT (Alneberg, J. et al. Nat Methods 11, 1144-1146, doi:10.1038/nmeth.3103 (2014)). After manual curation of the bins, a recommended step in the Anvi'o pipeline, MAGs were defined as bins with >70% completion in <10% redundancy. Dereplication of the MAGs from all profiles was done using fastANl (Jain, C., et al., Nat Commun 9, 5114, doi:10.1038/s41467-018-07641-9 (2018)) (97% similarity and k-mer size of 10). Gene calls with KEGG annotations were extracted from the non-redundant MAGs collection to form the gene catalog. Then metagenomic samples from the human cohort were pre-processed in a similar way to the murine metagenomic data, with the exception of using reference hg19 for host reads removal. Reads were then subsampled to an even depth of 8 M reads and samples of shallower sequencing depth were omitted from the analysis, resulting in a cohort of 60 human subjects. Finally, metagenomic profiles of the human samples were obtained by mapping the reads against the gene catalog described above using bowtie2. Samples were mean aggregated per subject to form a single metagenome for each participant. A binary classifier was trained by fitting a GradientBoosting classifier on top of partial least squares regression (both implemented in scikit-learn Python package, version 0.20.0) with default hyper-parameters. The ROC curve was evaluated by executing stratified k-fold iterations for six different splits.

RNA Sequencing. Library preparation was based on previously published protocol¹²², using RNase H (New England Biolabs, M0297) to selectively deplete target ribosomal RNA (rRNA). Specifically, we used a pool of 50-bp single-stranded DNA-oligonucleotides complementary to the murine rRNA 18S and 28S, which were mixed with equimolar concentrations. Total RNA (100-1,000 ng in 10 μl H2O) was mixed with an equal amount of rRNA oligo pool, diluted to 2 μl, and 3 μl 5× rRNA hybridization buffer (0.5 M Tris-HCl, 1 M NaCl, titrated with HCl to pH 7.4) was added. The hybridization mix was incubated at 95° C. for 2 min, then the temperature was slowly ramped (−0.1° C./s) to 37° C. In the meanwhile, the digestion-mix was prepared by adding 2 uL of RNase H to 2 μl RNase H Buffer and 1 μl H2O and further pre-heated at 37° C. As soon as the hybridization step reached 37° C., the mix was added and incubated at 37° C. for additional 30 min. The RNA was purified with 2.2× SPRI beads (AMPure XP, Beckmann Coulter), according to the manufacturer's instructions. The residual DNA-oligos were degraded by DNAse treatment (Thermo Fisher Scientific, AM2238), incubating the samples at 37° C. for 30 min with 5 μl DNase reaction mix (1 μl Turbo DNase, 1.25 μl Turbo DNase 10× buffer). The purification step with the 2.2× SPRI beads was repeated and suspended in 3.6 μl priming mix (0.3 μl random primers from New England

Biolabs, E7420, 3.3 μl H2O). Subsequently, primers were primed at 65° C. for 5 min and placed on ice. For subsequent first-strand cDNA synthesis, 2 μl of the first strand mix were added (1 μl 5× first strand buffer, NEB E7420; 0.125 μl RNase inhibitor, NEB E7420; 0.25 μl ProtoScript II reverse transcriptase, NEB E7420; and 0.625 μl of 0.2 μg/ml actinomycin D, Sigma-Aldrich, A1410) and the first strand synthesis and all subsequent library preparation steps were performed using NEBNext Ultra Directional RNA Library Prep Kit for Illumina (New England Biolabs, E7420) according to the manufacturer's instructions (all reaction volumes reduced to a quarter) and sequenced using the Illumina NextSeq platform. Reads containing adapters and low-quality reads were removed.

RNA Sequencing Analysis. Reads were aligned to the mm10 reference genome (UCSC) using STAR. Alignments were binned to genes using htseq-count¹²³. For each comparison, genes with read count ≥10⁻⁴ out of total reads and expressed in at least a fifth of a group between in each comparison were included in the analysis. Differentially expressed genes were found using DESeq2. Genes with padj <=0.05 were considered as differentially expressed. Heat maps were created using the regularized log transform (rlog) function of the DESeq2 package.

qPCR. DNA templates were diluted to a final amount of 1 ng per reaction. Amplifications were performed with the primer sets: BHMT forward, 5′-GCCACCGGCTTCAGAAAAA-3′ SEQ ID NO: 3; BHMT reverse, 5′CCGGAAGCTATTCGCAGATT-3′ SEQ ID NO: 4. HPRT forward, 5′-TCAGTCAACGGGGGACATAAA -3′ SEQ ID NO: 5; HPRT reverse, 5′-GGGGCTGTACTGCTTAACCAG -3′ SEQ ID NO: 6. Using the Fast SYBR™ Green Master Mix (ThermoFisher) in duplicates. Amplification conditions were: denaturation 95° C. for 20 s, followed by 40 cycles of denaturation 95° C. for 1 s; annealing 60° C. for 20 s followed by melting curve. Duplicates with >1 cycle difference were excluded from the analysis. Data were analyzed using the ΔΔCt method.

Targeted Metabolomics. D4-nicotinamide (50 ng ml-1; Cambridge Isotope Laboratories) was added to all samples as an internal standard. LC-MS/MS analysis was performed on Acquity UPLC system and triple quadrupole Xevo TQ-S (both Waters). TargetLynx (Waters) was applied for quantitation based on standard curves. Stool samples were weighed into 2-ml safe-lock Eppendorf tubes and 300 ul of 70% ethanol in DDW was added. Samples were homogenized using a beadbeater with metal balls. Serum samples (10 uL) were mixed with 90 uL of 70% acetonitrile. The extracts were centrifuged and filtered through PTFE 0.2-μm filter vials (Thompson) for analysis of dimethylglycine, trimethylglycine, and choline. For analysis of nicotine and cotinine, the samples were dried in a speed vac to remove the methanol before drying was completed in a lyophilizer, then re-dissolved in 100 μl of 20% ethanol, centrifuged, and filtered through PVDF 0.2-μm filters (Millex-GV, Millipore).

Nicotine and Cotinine: LC parameters: Acquity BEH C8 column (2.1×100 mm, 1.7 p.m; Waters) at 30° C. Gradient conditions of mobile phases A—10 mM ammonium formate, pH3.0 and B—acetonitrile: 0 min, 5% B; 2.5 min, 45% B; 2.8 min, 100% B; 3.3 min, 5% B; 5 min, 5% B. Injection volume 1.0 μl, flow rate 0.2 ml min-1. MS/MS parameters: electrospray ionization in positive-ion mode, desolvation temperature, 400° C.; desolvation gas flow, 700 1 h-1 ; cone gas flow, 150 1 h-1 ; nebulizer pressure, 7 Bar; capillary voltage, 0.5 kV, cone voltage 25 V. The MRM transitions - nicotine: 163.1>130.1 and 163.1>132.1, collision energy (CE) 16 and 12 V, respectively; cotinine: 177.1>80.0 and 177.1>98.1, CE 18 for both; d4-nicotinamide: 127 >81 and 127 >84, CE 19 and 17 V, respectively, with argon 0.10 mg min-1 as the collision gas.

Dimethylglycine, trimethylglycine, and choline: LC parameters: Cortecs HILIC column (2.1×100 mm, 1.6 μm; Waters) at 25° C. Gradient conditions of mobile phases A—15 mM ammonium formate, pH3.5 and B—acetonitrile: 0 min, 75% B; 0.5 min, 75% B; 3.5 min, 20% B; 3.6 min, 75% B; 6 min, 75% B. Injection volume 1.0 μl, flow rate 0.3 ml min-1. MS/MS parameters: electrospray ionization in positive-ion mode, desolvation temperature, 400° C.; desolvation gas flow, 800 1 h-1 ; cone gas flow, 150 1 h-1 ; nebulizer pressure, 7 Bar; capillary voltage, 2.8 kV, cone voltage 17 V. The MRM transitions- dimethylglycine: 104.0 >58.1, CE 11 V; trimethylglycine: 118.1>59.2, CE 15 V; choline: 104.2>60.2, CE 14 V; d4-nicotinamide: ibid., with argon 0.10 mg min-1 as the collision gas.

Metabolomics Profiling. Samples were collected, snap-frozen in liquid nitrogen and stored at −80° C. Sample preparation and analysis were performed by Metabolon Inc. Samples were prepared using the automated MicroLab STAR system (Hamilton). To remove protein, dissociate small molecules bound to protein or trapped in the precipitated protein matrix, and to recover chemically diverse metabolites, proteins were precipitated with methanol. The resulting extract was divided into five fractions: UPLC-MS/MS with positive ion mode electrospray ionization; UPLC-MS/MS with negative ion mode electrospray ionization; LC polar platform; GC-MS; and one sample was reserved for backup. Samples were placed briefly on a TurboVap (Zymark) to remove the organic solvent. For LC, the samples were stored overnight under nitrogen before preparation for analysis. For GC, each sample was dried under vacuum overnight before preparation for analysis.

Data extraction and compound identification- raw data was extracted, peak-identified and QC processed using Metabolon's hardware and software. Compounds were identified by comparison to library entries of purified standards or recurrent unknown entities. Administration of Metabolites. For the in vivo administration of DMG (N,N-Dimethylglycine, ≥99%, Sigma-Aldrich), Alzet osmotic minipumps model 2004 were used (infusing the compound at a rate of 0.25 μl per h for 28 days). The pumps were filled with 200 μl DMG (100mg /kg/day) diluted in PBS (-,-). Vehicle control pumps contained an equivalent volume of PBS (-,-). Mice were anesthetized by i.p. injection of ketamine (100 mg/kg) and xylazine (10 mg/kg) mixture, thereafter the skin of the neck was shaved and sterilized with 70% ethanol, a 1-cm incision was made in the skin, and the osmotic minipumps were inserted after minimal blunt dissection and placed above the right hind flank. The cut was then closed with sterile surgical clips and the mice were carefully monitored for any signs of stress, bleeding, pain, or abnormal behavior. For the in vivo administration of Nicotine, mice were administrated for 3 weeks with 0.15 mg/ml Nicotine by bidaily IP.

Human trial: Data of the observational human cohort were obtained from baseline samples of an unrelated experiment, approved by the Weizmann Institute of Science Bioethics and Embryonic Stem Cell Research oversight committee (IRB approval number 170-2). Written informed consent for this purpose was obtained from all subjects. Ninety-six healthy volunteers were recruited for this study. Age- and BMI-matched from a non-smoking (n=62) and an actively smoking (n=34) cohorts were utilized. Blood and stool samples were collected from the participants followed by targeted mass spectrometry and metagenomic sequencing respectively. All participants received financial compensation for their participation in the study. All subjects fulfilled the following inclusion criteria: males and females, aged 18-70, body mass index<28, ability to provide informed consent. Exclusion criteria included: (i) pregnancy or fertility treatments; (ii) usage of antibiotics or antifungals within 3 months prior to participation; (iii) consumption of probiotics or non-nutritive sweeteners within 1 month prior to participation, (iv) chronically active inflammatory, cardiovascular, infectious, endocrine or neoplastic disease within the three years prior to enrollment; (v) chronic gastrointestinal disorder, including inflammatory bowel disease and celiac disease, or gastrointestinal surgery such as bariatric surgery; (vi) neuropsychiatric disorder; (vii) coagulation disorders; (viii) pre-diagnosed type I or type II diabetes mellitus or treatment with anti-diabetic medications; (ix) alcohol or substance abuse. Adherence to inclusion and exclusion criteria was validated by medical doctors. po Statistical Analysis. All statistical tests were performed using GraphPad Prism8. Each treatment group consisted of at least two cages to control for ‘cage effect’. For pooled analysis of results from different independent repeats, all mice from the same experimental group were pooled and a new statistical comparison was made for the entire pooled experiment, as performed for the individual repeats. No intermediate statistical strategies were applied. For comparisons of 2 groups, unpaired t-test or Mann-Whitney U-test were performed. Wilcoxon tests were used to conduct pairwise comparisons. F- test was used for comparisons of more than 2 groups. For the correction of multiple comparisons test Sidak, Tukey, Dunn or false discovery rate (FDR) by Benjamini-Hochberg Procedure (BH) were performed. Non-parametric tests were used when the distribution was not known to be normal. In cases where missing values prevented usage of ANOVA with repeated measures, the analysis was done by fitting a linear mixed model as implemented in GraphPad Prism8. The linear mixed model uses a compound symmetry covariance matrix and fits using Restricted Maximum Likelihood (REML). The variances accounting for the models in smoking cessation were: weight change˜time*antibiotic*smoking (1| mouse). For the model in the GF experiments, the variances were: weight change˜time*period of sample acquisition*donor smoking status (1| mouse). The variances accounting for the model in the metabolomics analysis were: metabolites levels˜time*antibiotic*smoking (1| mouse). Hypothesis testing for taxonomic and functional microbiome composition data was done using permutational analysis of variance (PERMANOVA)¹²⁴, for comparisons between different time-points of the same group, the permutations were stratified only to those shuffling labels of samples from the same mouse. P-values of<0.05 were considered significant. *p<0.05; **p<0.01; ***p<0.001; ****p<0.0001. “ns.” symbol non-significant comparison.

### RESULTS

A smoking cessation-associated weight gain (SCWG) model in mice. To study possible microbiome impacts on smoking-related weight changes, specifically on SCWG, we utilized a mouse cigarette-smoking model, in which 10-week-old C57bl male mice were subjected to bi-daily cigarette smoke sessions in a whole-body exposure chamber, in which research cigarettes (64 research cigarettes per day) were consumed for a total period of 3 weeks (methods). Using this regimen, cotinine (nicotine's major breakdown product) serum levels were comparable to those measured in active human smokers⁷⁶. To induce obesogenic conditions favoring the induction of SCWG, all mice in all smoking experiments, unless otherwise specified, consumed a high-fat diet (HFD, 60 kcal % fat) starting 3 days prior to initiation of smoking exposure. The physiological impact of this smoking regimen on metabolic activity was quantified using intraperitoneal glucose tolerance tests and serum biochemistry measurements, including quantification of serum lipid profiles and hepatic aminotransferase activity proxying non-alcoholic fatty liver disease. To control for housing-related confounders, non-smoking littermate controls were subjected to the same confined smoking chamber environment, without the smoke inhalation, in all experiments. After three weeks of smoking, exposure to cigarette smoke was ceased in the ‘smoking’ (SMK) mouse group, while it actively continued in ‘ continuous smoking’ (continuous SMK) group. In key experiments, individual repeats were performed by two different investigators.

Indeed, as is observed in human smokers⁷⁷⁻⁸¹, active smoking was associated with a significant weight reduction in HFD-fed mice as compared to non-smoking HFD-fed controls, coupled with increased serum levels of Ammonia, and decreased Cholesterol and HDL levels, as previously described82-84. Smoking-induced weight reduction was mirrored by an improved glycemic response. Smoking cessation was associated with a rapid weight gain, recapitulating human SCWG, coupled with elevated serum activity of Aspartate aminotransferase (AST) and Alanine aminotransferase (ALT), compared to continuous smoking. Other glycemic-related features test and serum biochemistry measurements did not significantly change upon smoking cessation.

Smoking cessation-associated weight gain is associated with distinct metabolite alterations. The present inventors sought to unravel a mechanism by which an altered smoking-cessation-induced microbiome contributes to SCWG. To this aim, they performed a systemic serum and stool metabolomic profiling during active smoking (days 15 and 21, respectively) and smoking cessation (days 30 and 35, respectively, of HFD-consuming mice. To uncover microbiome-associated metabolites potentially contributing to SCWG, they employed a linear mixed model (methods) accounting for smoking, antibiotic and time, in search of serum metabolites whose differential levels paralleled the SCWG phenotype in the non-smoking and smoking mouse groups, and their respective antibiotics-treated groups. Using this model, they collectively identified 41 metabolites to be significantly impacted by smoking, antibiotics and their interactions (FIG. 1A).

A post-hoc hypothesis testing following the model further refined the candidate metabolites according to the following criteria: (i) significant differential expression between smoking and non-smoking mice; (ii) significant differential expression between smoking and antibiotic-treated smoking mice; (iii) insignificant differential expression between antibiotic-treated non-smoking mice and non-smoking mice; and (iv) insignificant differential expression between antibiotic-treated non-smoking mice and antibiotic-treated smoking mice. Collectively, the analysis revealed several common metabolites, potentially correlating with a microbiome-induced SCWG, hexadecadienoate (16:2n6), dimethylglycine, N-acetylglycine, 1-palmitoyl-2-gamma-linolenoyl-GPC (16:0/18:3n6) and Hexanoylglycine, as summarized in Table 1, herein below.

TABLE 1

| | Smoking | Antibiotic | Smoking:Antibiotic | SMK/NS |
|---|---|---|---|---|
| Metabolites | Main effect | Main effect | interaction | direction |
| Hexadecadienoate | 0.0019 | 0.011 | 7.18E−08 | Decreased |
| (16:2n6) | | | | |
| Dimethylglycine | 1.29E−06 | 0.035 | 0.0019 | Increased |
| N-acetylglycine | 0.0016 | 1.06E−05 | 0.0001 | Decreased |
| 1-palmitoyl-2- | 0.0011 | 4.61E−06 | 0.0013 | Decreased |
| gammalinolenoyl- | | | | |
| GPC(16:0/18:3n6) | | | | |
| Hexanoylglycine | 0.0052 | 0.0025 | 0.031 | Decreased |

Dimethylglycine (DMG) is a derivative of the amino acid glycine and was previously suggested to impact food absorption92. It may be synthesized from dietary choline sulfate, which is converted to choline, betaine aldehyde, betaine and DMG through a series of enzymatic steps involving the gut microbiome (choline sulfatase, choline dehydrogenase, betaine-aldehyde dehydrogenase) and either the microbiome or the mammalian liver (Betaine-Homocysteine S-Methyltransferase, BHMT⁹³). Indeed, inferring KEGG modules from the significantly differing KOs revealed that one of the differentially suggested modules was the one converting choline to betaine (M00555). Furthermore, fitting a generalized linear model (GLM) to model the dependency between weight changes occurring upon smoking cessation and EC levels measured during the smoking period, highlighted, among multiple functional microbiome features (FIG. 1B), a strong negative correlation between SCWG and smoking-period microbial betaine reductase (converting betaine to TMA, FIG. 1B, inset). Moreover, targeted mass spectroscopy analysis of stool samples obtained from GF mice receiving fecal microbiome transplantation from smoking-cessation donors revealed higher DMG levels as compared to those measured in GF mice receiving FMT from non-smoking controls (FIG. 1C).

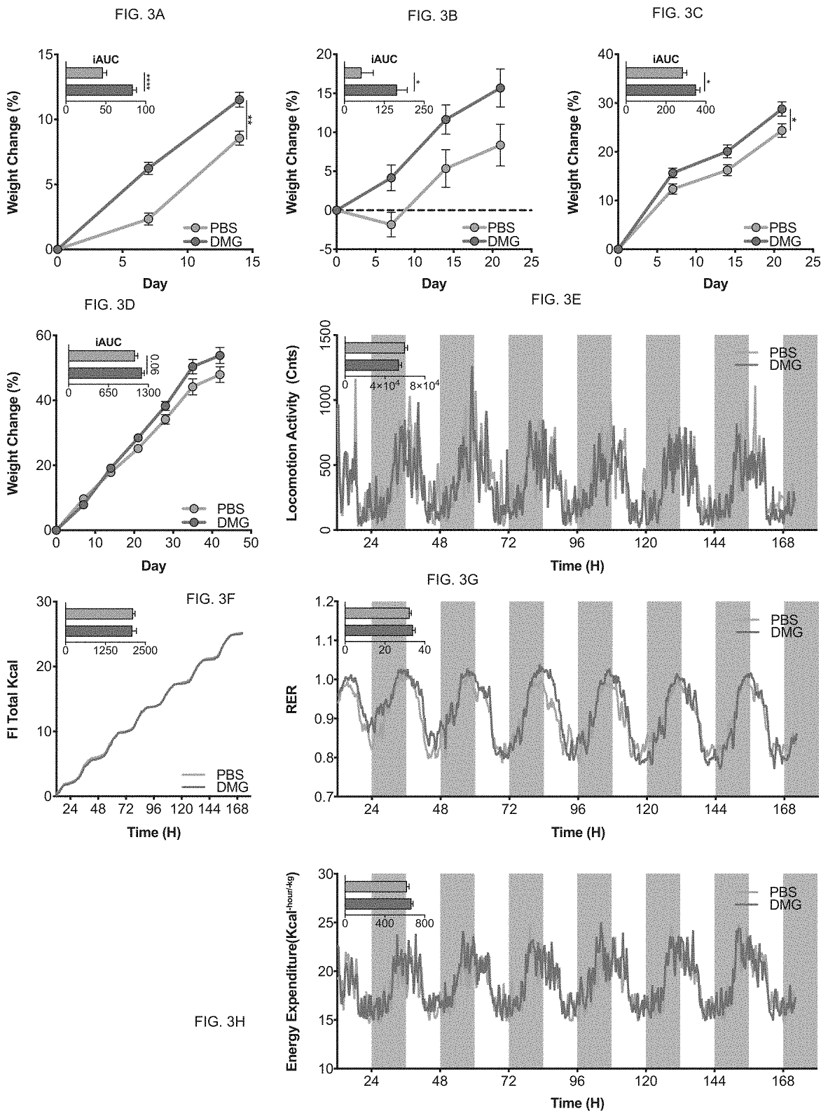

Importantly, the predicted levels of biosynthetic enzymes (PICRUSTt2, P542-PWY) of the choline to betaine pathway and the pathway's measured intermediary levels (Choline to Betaine) were altered in actively smoking mice (FIG. 1D). Additionally, a reduction in betaine reductase was observed downstream TMA to TMAO conversion step, collectively with the negative impact on SCWG (FIG. 1B), suggesting that reduced conversion of betaine to TMA-TMAO may further contribute to higher DMG levels noted during smoking (FIG. 1D). Importantly, the enzyme mediating the conversion step from Betaine to DMG was undetected in stool, but was significantly enhanced in its expression in the host's liver, as quantified by RNA sequencing (BHMT, FIG. 1D) and validated by quantitative PCR (BHMT). This concerted stepwise metabolic contribution of the gut microbiome and host liver jointly contributed to higher systemic DMG levels in the serum of smoking mice (FIG. 1D). These results were verified by targeted MS, demonstrating higher serum levels of betaine and DMG in smoking mice compared to non-smoking and antibiotics-treated smoking mice. In agreement, stool mass spectroscopy analysis demonstrated measurable DMG levels in colonized mice, but undetected levels in GF mice, corroborating the role of the microbiome in contributing to this biosynthetic pathway. DMG drives smoking cessation-associated weight gain by enhancing energy harvest. To determine whether DMG induces SCWG, naive, HFD-consuming, 10-week-old C57bl male mice were continuously administered, via osmotic pumps (methods), either DMG or vehicle (100 mg/kg/day) for 21 days. Importantly, DMG-treated mice featured a significant weight gain compared to vehicle-treated mice (FIG. 2A, pooled results, FIGS. 3A-D, 4 independent repeats).

Interestingly, metabolic features, including locomotion activity, total caloric uptake, RER and energy expenditure, were comparable between the mouse groups (FIGS. 3E-H). In contrast, quantification of residual caloric content in stool, inversely correlating with intestinal energy harvest, featured lower residual calories in DMG-supplemented mice, pointing to a higher gastrointestinal energy harvest capacity induced by DMG supplementation, that may result in weight gain (FIG. 2B).

The DMG-induced obesogenic effect was validated under SCWG conditions, by administrating DMG or vehicle to smoking-cessation mice treated with antibiotics and thus lacking this putative microbiome-derived SCWG-inducing metabolite. Of note, both DMG and vehicle supplementation were initiated prior to smoking cessation. Indeed, while smoking cessation in antibiotics-treated mice failed to induce SCWG, DMG supplementation restored SCWG despite microbiome depletion, with DMG-supplemented smoking-cessation mice reaching an indistinguishable weight compared to non-antibiotic treated ex-smoking mice, within days of supplementation (FIG. 2C, pooled results, FIGS. 4A-E, 3 independent repeats). The role of DMG as an inducer of SCWG was further validated by performing the smoking cessation experiment in mice consuming a choline-deficient diet (CDD). Under these conditions, the absence of the major DMG precursor was expected to result in lower DMG levels, thereby driving an attenuated SCWG. Indeed, serum DMG levels in all choline deficient-fed mice remained comparably low (FIG. 4F), with the smoking cessation group failing to develop SCWG (FIG. 2D, pooled results, FIGS. 4G-H, 2 independent repeats).

To further investigate the putative mechanisms by which DMG affects mice, characterization of transcriptomic signatures was performed using RNA-seq on epididymal fat (EAT), liver and gut (jejunum) samples obtained from HFD-fed non-smoking, smoking, and smoking-cessation mice, as well as HFD-fed, DMG- or vehicle supplemented mice. Interestingly in the liver, a positive correlation was noted between gene signatures corresponding to smoking and those corresponding to DMG supplementation (FIG. 4I, liver). Furthermore, signatures correlating to smoking coupled with antibiotics consumption were inversely correlated to those found upon DMG supplementation (FIG. 4I, liver). In contrast, no such correlations were observed in the epididimal adipose tissue (EAT) or intestinal samples (FIG. 4I, EAT and jejunumo, respectively). Together, these results suggest that DMG is a smoking-induced microbiome-derived metabolite that contributes to SCWG.

### Acetylglycine Features a Microbiome-Dependent Weight Lowering Activity.

N-acetylglycine (ACG, Aceturic acid), a derivative of the amino acid glycine, featured significantly lower levels among non-antibiotics-treated smoking and SCWG mice, as is highlighted by the present model (FIGS. 1A, 5C). Untargeted mass spectroscopy analysis of stool samples comparing SPF and GF mice showed significantly higher levels of ACG in SPF mice (FIG. 5D). To investigate the potential impact of ACG on SCWG, naive, HFD-consuming 10-week-old C57bl male mice were administered ACG incorporated into the diet (given its low water solubility; HFD containing 60 kcal % fat and 7500 mg N-Acetylglycine/kg Diet). Bomb-calorimetry confirmed that both HFD and ACG-HFD diets featured comparable caloric contents (FIG. 5E). Importantly, HFD-fed, ACG-treated mice featured a significantly lower weight gain compared to HFD-fed control mice (FIG. 2E, pooled results, FIGS. 5F-H, 3 independent repeats, and additionally an independent repeat in 7-week-old mice, FIG. 4I). This ACG-induced anti-obesogenic effect was further tested in SCWG conditions, by utilizing the SCWG model in non-antibiotics-treated, HFD-or HFD-ACG-fed mice. Indeed, while smoking cessation HFD-fed mice featured SCWG, ACG supplementation prevented SCWG under these conditions (FIG. 2F).

Together, these results suggest that smoking-, HFD-, and microbiome-induced elevation of DMG and depletion of ACG during smoking cessation in mice contribute to SCWG through induction of enhanced intestinal energy harvest. Microbiome depletion by antibiotic treatment or replenishment of altered metabolite levels may contribute to lower rates of SCWG.

### Metabolite Alterations in Human Smoking

The microbiome profile of a small cross-sectional age- and gender-matched cohort of 96 human individuals was analyzed (methods). To study the differences between smoking and non-smoking humans in light of the mouse model results, a metagenomic reference catalog of bacterial genomes was constructed which was assembled from metagenomes of mouse models (methods) and featuring a potential to drive the SCWG effect. Compared to non-smokers, fecal microbiomes from human smokers featured a distinct composition (NS=40, SMK=20, FIGS. 6B-C,) and KO abundance (FIGS. 6D-E). Binary classifier (methods) successfully discriminated human smokers and non-smokers based on microbiome taxonomic composition (FIG. 6F). Human smokers featured elevated levels of serum choline, betaine and DMG (NS=62, SMK=34, FIGS. 6G-I), similarly to smoking mice.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

### REFERENCES

1. Centers for Disease Control and Prevention (CDC). Vital signs: current cigarette smoking among adults aged ≥18 years with mental illness-United States, 2009-2011. MMWR. Morb. Mortal. Wkly. Rep. 62, 81-7 (2013).
2. WHO I Prevalence of tobacco smoking. WHO (2016).
3. Centers for Disease Control and Prevention (CDC). Cigarette smoking among adults-United States, 2006. MMWR. Morb. Mortal. Wkly. Rep. 56, 1157-61 (2007).
4. Tobacco in Western Pacific. Available at: www(dot)who.int/westernpacific/health-topics/tobacco. (Accessed: 3Apr. 2019)
5. Cancer Institute, N., Department of Health, U., Services, H. & Health Organization, W. Monograph 21. The Economics of Tobacco and Tobacco Control.
6. Health, N. C. for C. D. P. and H. P. (US) O. on S. and. The Health Consequences of Smoking-50 Years of Progress. The Health Consequences of Smoking-50 Years of Progress: A Report of the Surgeon General (Centers for Disease Control and Prevention (US), 2014).
7. (US), C. for D. C. and P., (US), N. C. for C. D. P. and H. P. & (US), O. on S. and H. How Tobacco Smoke Causes Disease: The Biology and Behavioral Basis for Smoking-Attributable Disease. How Tobacco Smoke Causes Disease: The Biology and Behavioral Basis for Smoking-Attributable Disease: A Report of the Surgeon General (Centers for Disease Control and Prevention (US), 2010).
8. (US), O. of the S. G. & (US), O. on S. and H. The Health Consequences of Smoking. The Health Consequences of Smoking: A Report of the Surgeon General (Centers for Disease Control and Prevention (US), 2004).
9. Hbejan, K. Smoking effect on ischemic heart disease in young patients. Heart Views 12, 1-6 (2011).
10. Dilic, M. et al. Cerebrovascular events: correlation with plaque type, velocity parameters and multiple risk factors. Med. Arh. 64, 204-7 (2010).
11. Aune, D., Schlesinger, S., Norat, T. & Riboli, E. Tobacco smoking and the risk of abdominal aortic aneurysm: a systematic review and meta-analysis of prospective studies. Sci. Rep. 8, 14786 (2018).
12. Lu, J. T. & Creager, M. A. The relationship of cigarette smoking to peripheral arterial disease. Rev. Cardiovasc. Med. 5, 189-93 (2004).
13. Buist, A. S., Vollmer, W. M. & McBurnie, M. A. Worldwide burden of COPD in high-and low-income countries. Part I. The burden of obstructive lung disease (BOLD) initiative. Int. J. Tuberc. Lung Dis. 12, 703-8 (2008).
14. Alsaadawi, T. Smoking is Associated with Increased Risk of Osteoporosis in Diabetes Mellitus Patients. J. Bone Reports Recomm. 02, (2016).
15. Seddon, J. M., Willett, W. C., Speizer, F. E. & Hankinson, S. E. A prospective study of cigarette smoking and age-related macular degeneration in women. JAMA 276, 1141-6 (1996).
16. Is nicotine addictive? | National Institute on Drug Abuse (NIDA). Available at: www(dot)drugabuse(dot)gov/publications/research-reports/tobacco-nicotine-e-cigarettes/nicotine-addictive. (Accessed: 3rd April 2019)
17. The ,Health Benefits of SMOKING CESSATION.
18. Centers for Disease Control and Prevention (CDC). Quitting smoking among adults--United States, 2001-2010. MMWR. Morb. Mortal. Wkly. Rep. 60, 1513-9 (2011).
19. Aubin, H.-J. Management of emergent psychiatric symptoms during smoking cessation. Curr. Med. Res. Opin. 25, 519-525 (2009).
20. Brown, R. A., Kahler, C. W., Zvolensky, M. J., Lejuez, C. . & Ramsey, S. E. Anxiety sensitivity: Relationship to negative affect smoking and smoking cessation in smokers with past major depressive disorder. Addict. Behav. 26, 887-899 (2001).
21. Siu, A. L. & U.S. Preventive Services Task Force. Behavioral and Pharmacotherapy Interventions for Tobacco Smoking Cessation in Adults, Including Pregnant Women: U.S. Preventive Services Task Force Recommendation Statement. Ann. Intern. Med. 163, 622-34 (2015).
22. Hughes, J. Effects of abstinence from tobacco: Valid symptoms and time course. Nicotine Tob. Res. 9, 315-327 (2007).
23. Lycett, D. et al. The association between smoking cessation and glycaemic control in patients with type 2 diabetes: a THIN database cohort study. Lancet Diabetes Endocrinol. 3, 423-430 (2015).
24. Hughes, J. R., Stead, L. F., Hartmann-Boyce, J., Cahill, K. & Lancaster, T. Antidepressants for smoking cessation. Cochrane Database Syst. Rev. (2014). doi:10.1002/14651858.CD000031.pub4
25. Tian, J., Venn, A., Otahal, P. & Gall, S. The association between quitting smoking and weight gain: a systemic review and meta-analysis of prospective cohort studies. Obes. Rev. 16, 883-901 (2015).
26. Dhurandhar, E. J. & Keith, S. W. The aetiology of obesity beyond eating more and exercising less. Best Pract. Res. Clin. Gastroenterol. 28, 533-544 (2014).
27. Aubin, H.-J., Farley, A., Lycett, D., Lahmek, P. & Aveyard, P. Weight gain in smokers after quitting cigarettes: meta-analysis. BMJ 345, e4439 (2012).
28. Harris, K. K., Zopey, M. & Friedman, T. C. Metabolic effects of smoking cessation. Nat. Rev. Endocrinol. 12, 299-308 (2016).
29. Biedermann, L. et al. Smoking Cessation Induces Profound Changes in the Composition of the Intestinal Microbiota in Humans. PLoS One 8, e59260 (2013).
30. Rodin, J. Weight change following smoking cessation: the role of food intake and exercise. Addict. Behay. 12, 303-17 (1987).
31. Moffatt, R. J. & Owens, S. G. Cessation from cigarette smoking: changes in body weight, body composition, resting metabolism, and energy consumption. Metabolism. 40, 465-70 (1991).
32. Stamford, B. A., Matter, S., Fell, R. D. & Papanek, P. Effects of smoking cessation on weight gain, metabolic rate, caloric consumption, and blood lipids. Am. J. Clin. Nutr. 43, 486-494 (1986).
33. Ferrara, C., Kumar, M., Nicklas, B., McCrone, S. & Goldberg, A. Weight gain and adipose tissue metabolism after smoking cessation in women. Int. J. Obes. 25, 1322-1326 (2001).
34. Dallosso, H. M. & James, W. P. The role of smoking in the regulation of energy balance. Int. J. Obes. 8, 365-75 (1984).
35. Perkins, K. A., Epstein, L. H., Marks, B. L., Stiller, R. L. & Jacob, R. G. The Effect of Nicotine on Energy Expenditure during Light Physical Activity. N. Engl. J. Med. 320, 898-903 (1989).
36. Martinez de Morentin, P. B. et al. Nicotine Induces Negative Energy Balance Through Hypothalamic AMP-Activated Protein Kinase. Diabetes 61, 807-817 (2012).
37. Chen, H. et al. Long-term cigarette smoke exposure increases uncoupling protein expression but reduces energy intake. Brain Res. 1228, 81-88 (2008).
38. Audrain-McGovern, J. & Benowitz, N. L. Cigarette Smoking, Nicotine, and Body Weight. Clin. Pharmacol. Ther. 90, 164-168 (2011).
39. Mineur, Y. S. et al. Nicotine Decreases Food Intake Through Activation of POMC Neurons. Science (80-.). 332, 1330-1332 (2011).
40. Levine, M. D., Cheng, Y., Kalarchian, M. A., Perkins, K. A. & Marcus, M. D. Dietary intake after smoking cessation among weight-concerned women smokers. Psychol. Addict. Behay. 26, 969-973 (2012).
41. Hur, Y.-N., Hong, G.-H., Choi, S.-H., Shin, K.-H. & Chun, B.-G. High fat diet altered the mechanism of energy homeostasis induced by nicotine and withdrawal in C57BL/6 mice. Mol. Cells 30, 219-226 (2010).
42. Pepino, M. Y. & Mennella, J. A. Cigarette smoking and obesity are associated with decreased fat perception in women. Obesity 22, 1050-1055 (2014).
43. Blum, K., Liu, Y., Shriner, R. & Gold, M. S. Reward circuitry dopaminergic activation regulates food and drug craving behavior. Curr. Pharrn. Des. 17, 1158-67 (2011).
44. Hankey, C. & Leslie, W. Is weight gain after smoking cessation an important concern? Nat. Rev. Endocrinol. 8, 630-632 (2012).
45. Sekirov, I., Russell, S. L., Antunes, L. C. M. & Finlay, B. B. Gut Microbiota in Health and Disease. Physiol. Rev. 90, 859-904 (2010).
46. Donia, M. S. & Fischbach, M. A. Small molecules from the human microbiota. Science (80-.). 349, (2015).
47. Gilbert, J. A. et al. Current understanding of the human microbiome. Nat. Med. 24, 392-400 (2018).
48. Cho, I. & Blaser, M. J. The human microbiome: At the interface of health and disease. Nature Reviews Genetics 13, 260-270 (2012).
49. Dominguez-Bello, M. G., Godoy-Vitorino, F., Knight, R. & Blaser, M. J. Role of the microbiome in human development. Gut 68, 1108-1114 (2019).
50. Dodd, D. et al. A gut bacterial pathway metabolizes aromatic amino acids into nine circulating metabolites. Nature 551, 648-652 (2017).
51. Moreno-Indias, I., Cardona, F., Tinahones, F. J. & Queipo-Ortuñlo, M. I. Impact of the gut microbiota on the development of obesity and type 2 diabetes mellitus. Front. Microbiol. 5, 190 (2014).
52. Ussar, S. et al. Interactions between Gut Microbiota, Host Genetics and Diet Modulate the Predisposition to Obesity and Metabolic Syndrome. Cell Metab. 22, 516-530 (2015).
53. Suez, J. et al. Artificial sweeteners induce glucose intolerance by altering the gut microbiota. Nature 514, 181-186 (2014).
54. Zhang, X. et al. Human Gut Microbiota Changes Reveal the Progression of Glucose Intolerance. PLoS One 8, e71108 (2013).
55. Safari, Z. & Gérard, P. The links between the gut microbiome and non-alcoholic fatty liver disease (NAFLD). Cell. Mol. Life Sci. 76, 1541-1558 (2019).
56. Tripathi, A. et al. The gut-liver axis and the intersection with the microbiome. Nature Reviews Gastroenterology and Hepatology 15, 397-411 (2018).
57. Tang, W. H. W. & Hazen, S. L. The Gut Microbiome and Its Role in Cardiovascular Diseases. Circulation 135, 1008-1010 (2017).
58. Aron-Wisnewsky, J., Doré, J. & Clement, K. The importance of the gut microbiota after bariatric surgery. Nat. Rev. Gastroenterol. Hepatol. 9, 590-598 (2012).
59. Shen, J., Obin, M. S. & Zhao, L. The gut microbiota, obesity and insulin resistance. Mol. Aspects Med. 34, 39-58 (2013).
60. Turnbaugh, P. J. et al. An obesity-associated gut microbiome with increased capacity for energy harvest. Nature 444, 1027-1031 (2006).
61. Conterno, L., Fava, F., Viola, R. & Tuohy, K. M. Obesity and the gut microbiota: does up-regulating colonic fermentation protect against obesity and metabolic disease? Genes Nutr. 6, 241-260 (2011).
62. Khan, M. J., Gerasimidis, K., Edwards, C. A. & Shaikh, M. G. Role of Gut Microbiota in the Aetiology of Obesity: Proposed Mechanisms and Review of the Literature. J. Obes. 2016, 1-27 (2016).
63. López, M. EJE PRIZE 2017: Hypothalamic AMPK: a golden target against obesity? Eur. J. Endocrinol. 176, R235-R246 (2017).
64. Hardie, D. G. AMPK: a key regulator of energy balance in the single cell and the whole organism. Int. J. Obes. 32, S7-S12 (2008).
65. Bäckhed, F. et al. The gut microbiota as an environmental factor that regulates fat storage. 101, 15718-15723 (2004).
66. Pindjakova, J. et al. Gut Dysbiosis and Adaptive Immune Response in Diet-induced Obesity vs. Systemic Inflammation. Front. Microbiol. 8, 1157 (2017).
67. Cani, P. D. et al. Metabolic Endotoxemia Initiates Obesity and Insulin Resistance. Diabetes 56, 1761-1772 (2007).
68. Vijay-Kumar, M. et al. Metabolic syndrome and altered gut microbiota in mice lacking Toll-like receptor 5. Science 328, 228-31 (2010).
69. Stewart, C. J. et al. Effects of tobacco smoke and electronic cigarette vapor exposure on the oral and gut microbiota in humans: a pilot study. PeerJ 6, e4693 (2018).
70. Lee, S. H. et al. Association between Cigarette Smoking Status and Composition of Gut Microbiota: Population-Based Cross-Sectional Study. J. Clin. Med. 7, (2018).
71. Shanahan, E. R. et al. Influence of cigarette smoking on the human duodenal mucosa-associated microbiota. Microbiome 6, 150 (2018).
72. Biedermann, L. et al. Smoking Cessation Alters Intestinal Microbiota. Inflamm. Bowel Dis. 20, 1496-1501 (2014).
73. Wang, H. et al. Side-stream smoking reduces intestinal inflammation and increases expression of tight junction proteins. World J. Gastroenterol. 18, 2180 (2012).
74. Savin, Z., Kivity, S., Yonath, H. & Yehuda, S. Smoking and the intestinal microbiome. Arch. Microbiol. 200, 677-684 (2018).
75. Lim, M. Y. et al. Analysis of the association between host genetics, smoking, and sputum microbiota in healthy humans. Sci. Rep. 6, 23745 (2016).
76. Witschi, H., Espiritu, I., Dance, S. T. & Miller, M. S. A Mouse Lung Tumor Model of Tobacco Smoke Carcinogenesis. Toxicological Sciences 68, 322-330 (Narnia, 2002).
77. Morris, R. W. et al. Heavier smoking may lead to a relative increase in waist circumference: Evidence for a causal relationship from a Mendelian randomisation meta-analysis. The CARTA consortium. BMJ Open 5, (2015).
78. Williamson, D. F. et al. Smoking cessation and severity of weight gain in a national cohort. N. Engl. J. Med. 324, 739-745 (1991).
79. Sneve, M. & Jorde, R. Cross-sectional study on the relationship between body mass index and smoking, and longitudinal changes in body mass index in relation to change in smoking status: The Tromso Study. Scand. J. Public Health 36, 397-407 (2008).
80. Molarius, A., Seidell, J. C., Kuulasmaa, K., Dobson, A. J. & Sans, S. Smoking and relative body weight: An international perspective from the WHO MONICA project. J. Epidemiol. Community Health 51, 252-260 (1997).
81. Munafò, M. R., Tilling, K. & Ben-Shlomo, Y. Smoking status and body mass index: A longitudinal study. Nicotine Tob. Res. 11, 765-771 (2009).
82. Van Amsterdam, J. et al. Effect of ammonia in cigarette tobacco on nicotine absorption in human smokers. Food Chem. Toxicol. 49, 3025-3030 (2011).
83. Campbell, S. C., Moffatt, R. J. & Stamford, B. A. Smoking and smoking cessation-The relationship between cardiovascular disease and lipoprotein metabolism: A review. Atherosclerosis 201, 225-235 (2008).
84. He, B. M., Zhao, S. P. & Peng, Z. Y. Effects of cigarette smoking on HDL quantity and function: Implications for atherosclerosis. Journal of Cellular Biochemistry 114, 2431-2436 (2013).
85. Filozof, C., Fernández Pinilla, M. C. & Fernandez-Cruz, A. Smoking cessation and weight gain. Obesity Reviews 5, 95-103 (2004).
86. Bush, T., Lovejoy, J. C., Deprey, M. & Carpenter, K. M. The effect of tobacco cessation on weight gain, obesity, and diabetes risk. Obesity 24, 1834-1841 (2016).
87. Graff-Iversen, S., Hewitt, S., Forsén, L., Grotvedt, L. & Ariansen, I. Associations of tobacco smoking with body mass distribution; A population-based study of 65,875 men and women in midlife. BMC Public Health 19, 1439 (2019).
88. Taylor, A. E. et al. The effect of body mass index on smoking behaviour and nicotine metabolism: A Mendelian randomization study. Hum. Mol. Genet. 28, 1322-1330 (2019).
89. Piirtola, M. et al. Association of current and former smoking with body mass index: A study of smoking discordant twin pairs from 21 twin cohorts. PLoS One 13, 36 (2018).
90. Bäckhed, F., Ley, R. E., Sonnenburg, J. L., Peterson, D. A. & Gordon, J. I. Host-bacterial mutualism in the human intestine. Science 307, 1915-1920 (2005).
91. Langille, M. G. I. et al. Predictive functional profiling of microbial communities using 16S rRNA marker gene sequences. Nat. Biotechnol. 31, 814-821 (2013).
92. Prola, L. et al. Effects of N,N-dimethylglycine sodium salt on apparent digestibility, vitamin E absorption, and serum proteins in broiler chickens fed a high- or low-fat diet. Poult. Sci. 92, 1221-1226 (2013).
93. Teng, Y.-W., Mehedint, M. G., Garrow, T. A. & Zeisel, S. H. Deletion of betaine-homocysteine S-methyltransferase in mice perturbs choline and 1-carbon metabolism, resulting in fatty liver and hepatocellular carcinomas. J. Biol. Chem. 286, 36258-67 (2011).
94. Butte, N. F. et al. Global metabolomic profiling targeting childhood obesity in the Hispanic population. Am. J. Clin. Nutr. 102, 256-267 (2015).
95. Zhao, H. et al. Metabolomics-identified metabolites associated with body mass index and prospective weight gain among Mexican American women. Obes. Sci. Pract. 2, 309-317 (2016).
96. Wikoff, W. R. et al. Metabolomics analysis reveals large effects of gut microflora on mammalian blood metabolites. Proc. Natl. Acad. Sci. U. S. A. 106, 3698-3703 (2009).
97. Soares, P. N. et al. Cigarette smoke during breastfeeding in rats changes glucocorticoid and vitamin D status in obese adult offspring. Int. J. Mol. Sci. 19, (2018).
98. Khan, A. et al. Cigarette smoking blocks the benefit from reduced weight gain for insulin action by shifting lipids deposition to muscle. Clin. Sci. (Loud). 134, 1659-1673 (2020).
99. Fennema, D., Phillips, I. R. & Shephard, E. A. Trimethylamine and trimethylamine N-oxide, a Flavin-Containing Monooxygenase 3 (FMO3)-mediated host-microbiome metabolic axis implicated in health and disease. Drug Metabolism and Disposition 44, 1839-1850 (2016).
100. Wang, Z. et al. Gut flora metabolism of phosphatidylcholine promotes cardiovascular disease. Nature 472, 57-65 (2011).
101. Koeth, R. A. et al. Intestinal microbiota metabolism of 1-carnitine, a nutrient in red meat, promotes atherosclerosis. Nat. Med. 19, 576-585 (2013).
102. Thaiss, C. A. et al. Persistent microbiome alterations modulate the rate of post-dieting weight regain. Nature 540, 544-551 (2016).
103. Bolyen, E. et al. Reproducible, interactive, scalable and extensible microbiome data science using QIIME 2. Nature Biotechnology 37, 852-857 (2019).
104. Louca, S. & Doebeli, M. Efficient comparative phylogenetics on large trees. doi:10.1093/bioinformatics/btx701
105. Czech, L., Barbera, P. & Stamatakis, A. Genesis and Gappa: processing, analyzing and visualizing phylogenetic (placement) data. doi:10.1093/bioinformatics/btaa070
106. Barbera, P. et al. EPA-ng: Massively Parallel Evolutionary Placement of Genetic Sequences. Syst. Biol 68, 365-369 (2019).
107. Ye, Y. & Doak, T. G. A Parsimony Approach to Biological Pathway Reconstruction/Inference for Genomes and Metagenomes. PLoS Comput. Biol. 5, e1000465 (2009).
108. Douglas, G. M. et al. PICRUSt2: An improved and extensible approach for metagenome inference. bioRxiv 672295 (2019). doi:10.1101/672295
109. Bolger, A. M., Lohse, M. & Usadel, B. Genome analysis Trimmomatic: a flexible trimmer for Illumina sequence data. 30, 2114-2120 (2014).
110. Wood, D. E., Lu, J. & Langmead, B. Improved metagenomic analysis with Kraken 2. Genome Biol. (2019). doi:10.1186/s13059-019-1891-0
111. Lu, J., Breitwieser, F. P., Thielen, P. & Salzberg, S. L. Bracken: Estimating species abundance in metagenomics data. PeerJ Comput. Sci. (2017). doi:10.7717/peerj-cs.104
112. Buchfink, B., Xie, C. & Huson, D. H. Fast and sensitive protein alignment using DIAMOND. Nature Methods (2014). doi:10.1038/nmeth.3176
113. Eren, A. M. et al. Anvi'o: An advanced analysis and visualization platformfor ‘omics data. PeerJ (2015). doi:10.7717/peerj.1319
114. Delmont, T. O. et al. Nitrogen-fixing populations of Planctomycetes and Proteobacteria are abundant in surface ocean metagenomes. Nat. Microbiol. 3, 804-813 (2018).
115. Li, D., Liu, C. M., Luo, R., Sadakane, K. & Lam, T. W. MEGAHIT: An ultra-fast single-node solution for large and complex metagenomics assembly via succinct de Bruijn graph. Bioinformatics (2015). doi:10.1093/bioinformatics/btv033
116. Eddy, S. R. Accelerated profile HMM searches. PLoS Comput. Biol. (2011). doi:10.1371/journal.pcbi.1002195
117. Kim, D., Song, L., Breitwieser, F. P. & Salzberg, S. L. Centrifuge: Rapid and sensitive classification of metagenomic sequences. Genome Res. (2016). doi:10.1101/gr.210641.116
118. Langmead, B. & Salzberg, S. Bowtie2. Nat. Methods (2013). doi:10.1038/nmeth.1923.Fast
119. Alneberg, J. et al. Binning metagenomic contigs by coverage and composition. Nat. Methods 11, 1144-1146 (2014).
120. Jain, C., Rodriguez-R, L. M., Phillippy, A. M., Konstantinidis, K. T. & Aluru, S. High throughput ANI analysis of 90K prokaryotic genomes reveals clear species boundaries. Nat. Commun. (2018). doi:10.1038/s41467-018-07641-9
121. Anders, S. & Huber, W. Differential expression analysis for sequence count data. Genome

Biol. 11, 1-12 (2010).
122. Blacher, E. et al. Potential roles of gut microbiome and metabolites in modulating ALS in mice. doi:10.1038/s41586-019-1443-5
123. Anders, S., Pyl, P. T. & Huber, W. HTSeq-A Python framework to work with high-throughput sequencing data. Bioinformatics (2015). doi:10.1093/bioinformatics/btu638
124. Anderson, M. J. A new method for non-parametric multivariate analysis of variance. Austral Ecol. 26, 32-46 (2001).

In the claims:

1. A method of treating obesity in a subject in need thereof, the method comprising administering to the subject a therapeutically effective amount of an agent that specifically increases the amount of hexadecadienoate (16:2n6), N-acetylglycine, 1-palmitoyl-2-gamma-linolenoyl-GPC (16:0/18:3n6) and/or Hexanoylglycine; or an agent that decreases the amount of dimethylglycine (DMG) in the fecal metabolome of the subject, thereby treating the obesity.

2. A method of reducing the risk of weight gain following nicotine smoking cessation in a subject comprising administering to the subject an effective amount of an agent that specifically increases the amount of hexadecadienoate (16:2n6), N-acetylglycine, 1-palmitoyl -2-gamma-linolenoyl-GPC (16:0/18:3n6) and/or Hexanoylglycine; or an agent that decreases the amount of dimethylglycine (DMG) in the fecal metabolome of the subject, thereby reducing the risk of weight gain in the subject.

3. The method of claim 1, wherein the agent comprises hexadecadienoate (1:2n6), N-acetylglycine, 1-palmitoyl-2-gamma-linolenoyl-GPC (16:0/18:3n6) and/or Hexanoylglycine.

4. The method of claim 1, wherein the agent that decreases the amount of DMG is an inhibitor of the DMG synthesis pathway.

5. The method of claim 1, wherein the agent that decreases the amount of DMG comprises a choline-poor diet.

6. The method of claim 2, wherein said administering is effected immediately following smoking cessation.

7. A method of reducing the risk of weight gain following nicotine smoking cessation in a subject comprising administering to the subject an effective amount of a fecal transplant derived from a healthy, non-smoker, thereby reducing the risk of weight gain in the subject.

8-23. (canceled)

24. The method of claim 2, wherein the agent comprises hexadecadienoate (16:2n6), N-acetylglycine, 1-palmitoyl-2-gamma-linolenoyl-GPC (16:0/18:3n6) and/or Hexanoylglycine.

25. The method of claim 2, wherein the agent that decreases the amount of DMG is an inhibitor of the DMG synthesis pathway.

26. The method of claim 2, wherein the agent that decreases the amount of DMG comprises a choline-poor diet.
